(19) United States
(12) Patent Application Publication
(10) Pub. No.: US 2026/0097234 A1
(43) Pub. Date: Apr. 9, 2026

(54) SYSTEMS AND METHODS FOR GENERATING DATA STRUCTURES USING EVENT DATA FROM DISPARATE SOURCES

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Doori Rose, New York, NY (US); Laura Cervino, Rye, NY (US); Joseph Deasy, New York, NY (US); Sharif Elguindi, New York, NY (US); Anyi Li, Scarsdale, NY (US); Jean Moran, Ann Arbor, MI (US); Gregory Niyazov, New York, NY (US); Ellen Pinto, New York, NY (US); Lakshmi Santana, New York, NY (US); Darian Pazgan-Lorenzo, Berea, OH (US)

(21) Appl. No.: 19/352,360

(22) Filed: Oct. 7, 2025

Related U.S. Application Data
(60) Provisional application No. 63/704,803, filed on Oct. 8, 2024.

(51) Int. Cl. A61N 5/10; G06N 3/0475; G06T 7/00; G16H 40/20

(52) U.S. Cl. CPC A61N 5/1031; A61N 5/1039; A61N 5/1064; A61N 5/1071; G06N 3/0475; G06T 7/0012; G16H 40/20; G06T 2207/10081; G06T 2207/10088; G06T 2207/10104; G06T 2207/10116

(57) ABSTRACT

Presented herein are systems and methods for generating data structures for data structures for events detected across data sources in network environments. A computing system may maintain, on a data repository, a profile for a subject at risk of or diagnosed with cancer.

The profile may identify a plurality of event identifiers for a corresponding plurality of events associated with administration of radiotherapy to the subject. The computing system may apply a prompt based on a request and at least a portion of the profile to a generative machine learning (ML) model. The computing system may generate, based on applying the prompt to the generative ML model, a data structure comprising (i) a plurality of nodes corresponding to the respective plurality of event identifiers and (ii) a plurality of edges each defining a relationship between a corresponding pair of the plurality of nodes.

### CROSS REFERENCE TO RELATED APPLICATIONS

The present application claims the benefit of priority under 35 U.S.C. § 119(e) to U.S. Provisional Ser. No. 63/704,803 , filed Oct. 8, 2024, which is incorporated herein by reference in its entirety.

### BACKGROUND

A computer system may apply a machine learning model on an input dataset to generate an output.

### SUMMARY

Aspects of the present disclosure are directed to systems, methods, and non-transitory computer readable media for generating data structures for data structures for events detected across data sources in network environments. One or more processors coupled with memory may detect, from one or more data sources in a network environment, an event associated with provision of radiation to a subject. The one or more processors may update, using an event identifier and a timestamp corresponding to the event, a record comprising (i) a plurality of event identifiers for a corresponding plurality of events associated with the subject and (ii) a plurality of timestamps corresponding to the plurality of events on a database. The one or more processors may generate a prompt based on at least a portion of the record on the database. The one or more processors may provide the prompt to a generative machine learning (ML) model, wherein the generative ML model may be established using a plurality of corpuses, each of the plurality of corpuses including: (i) a respective record of a corresponding subject identifying (a) a respective plurality of event identifiers for a respective plurality of events associated with provision of radiation in the corresponding subject and (b) a respective plurality of timestamps corresponding to the respective plurality of event identifiers, and (ii) a respective data structure identifying (a) a plurality of nodes corresponding to the respective plurality of event identifiers and (b) a respective plurality of edges each defining a relationship between a corresponding pair of the plurality of nodes. The one or more processors may generate, based on applying the prompt to the generative ML model, a data structure comprising (i) a plurality of nodes corresponding to a plurality of event identifiers and (ii) a plurality of edges each defining a relationship between a corresponding pair of the plurality of nodes. The one or more processors may provide, via a user interface, an output based on the data structure for the subject.

In some embodiments, the one or more processors may receive, via the user interface, a report comprising at least one of (i) a plurality of radiation parameters defining the provision of the radiation to an organ of the subject, wherein the plurality of radiation parameters associated with the radiation comprises at least one of a target volume, a dose of radiation, a dose distribution, a beam configuration, or a radiation type, wherein the radiation comprises at least one of intensity-modulated radiation therapy (IMRT), external beam radiation therapy (EBRT), stereotactic body radiation therapy (SBRT), image-guided radiation therapy (IGRT), or brachytherapy; or (ii) a plurality of characteristics defining the condition in the subject, wherein the plurality of characteristics defining cancer in the subject comprises at least one of a cancer type, a tumor classification, a tumor size, a tumor appearance, or a tumor grade. The one or more processors may update the record for the subject on the database using the report.

In some embodiments, the one or more processors may retrieve, from one or more data sources, data associated with the plurality of event identifiers for the corresponding plurality of events and a corresponding plurality of timestamps. The plurality of events may include at least one of approval of radiation, generation of radiation simulation, provision of radiation, subject diagnosis, or an acquisition of a biomedical image. The biomedical image may be in accordance with one of a plurality of imaging modalities including a whole slide imaging (WSI) modality, a computed tomography (CT) modality, a magnetic resonance imaging (MRI) modality, a positron emission tomography (PET) modality, or an x-ray imaging modality. The one or more processors may generate, for storage on the database, the record, in accordance with a template based on the data retrieved from the one or more data sources.

In some embodiments, the one or more processors may provide, for presentation, the user interface comprising one or more user interface elements to select at least one of a plurality of radiation plans for the subjects. Each of the plurality of radiation plans may identify a corresponding plurality of radiation parameters defining administration of a respective radiation to an organ in the subject at a corresponding time. The one or more processors may select, for presentation via the user interface, a second radiation plan from the plurality of radiation plans based on interaction with the one or more user interface elements.

In some embodiments, the one or more processors may generate the data structure defining a timeline to include the plurality of nodes corresponding to the respective plurality of event identifiers, each of the plurality of nodes configured to provide information on a corresponding event of the plurality of events responsive to interaction. In some embodiments, the one or more processors may generate a second prompt in accordance with a template for a user type of the user interface and using at least a portion of the record for the subject. The one or more processors may provide the second prompt to the generative ML model; to generate a report identifying at least one of (i) one or more of the plurality of events corresponding to the plurality of event identifiers (ii) a plurality of therapy parameters defining provision of radiation to an organ in the subject or (iii) a plurality of characteristics defining cancer in the subject. The one or more processors may provide, via the user interface, a second output based on the report.

In some embodiments, the one or more processors may identify, for provision of the radiation to the subject, a plurality of calendars for a plurality of clinicians. Each calendar of the plurality of calendars may identify a plurality of time slots indicating as one of available or unavailable for a corresponding clinician of the plurality of clinicians. The one or more processors may generate for presentation via the user interface, assignment availability information based on the plurality of calendars for a plurality of clinicians. In some embodiments, the one or more processors may generate, using a radiation plan corresponding to at least one of the plurality of events, a graph to identify one or more dosage values defining the provision of the radiation for each volume of a plurality of volumes in an organ of the subject.

In some embodiments, the one or more processors may maintain the record to include a plurality of messages associated with the provision of the radiation to the subject from one or more data sources. The one or more processors may receive, from a first client device in the network environment, a message associated with the provision of the radiation to the subject. The one or more processors may provide, to a second client device for presentation, a notification identifying the message. In some embodiments, the record may include at least one of (i) a report including a first plurality of radiation parameters defining radiation administered to an organ in the subject, or (ii) a simulated radiation plan including a second plurality of radiation parameters defining provision of radiation to the organ in the subject. In some embodiments, the subject may be at risk of or diagnosed with cancer comprising at least one of lung cancer, brain cancer, head and neck cancer, colon cancer, rectal cancer, uterine cancer, endometrial cancer, stomach cancer, ovarian cancer, cervical cancer, bladder cancer, or breast cancer.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a block diagram of a system for managing radiotherapy informatics for subjects using data of disparate modalities in accordance with an illustrative embodiment.

FIG. 2 depicts a block diagram of a process to collect data in various modalities in the system for managing radiotherapy informatics in accordance with an illustrative embodiment.

FIG. 3 depicts a block diagram of a process to apply generative machine learning (ML) models to the data in the system for managing radiotherapy informatics in accordance with an illustrative embodiment.

FIG. 4 depicts a block diagram of a process to produce outputs regarding radiotherapy plans in the system for managing radiotherapy informatics in accordance with an illustrative embodiment.

FIG. 5 depicts an example screenshot of a user interface for presenting a timeline of clinical events associated with administration of radiotherapy to a subject in accordance with an illustrative embodiment.

FIG. 6 depicts an example screenshot of a user interface for presenting medical records associated with administration of radiotherapy to a subject in accordance with an illustrative embodiment.

FIG. 7 depicts an example screenshot of a user interface for presenting assignment of personnel for the administration of radiotherapy to a subject in accordance with an illustrative embodiment.

FIG. 8 depicts an example screenshot of a user interface for presenting analytics associated with the administration of radiotherapy to a subject in accordance with an illustrative embodiment.

FIG. 9 depicts an example screenshot of a user interface for presenting plan checker dashboard associated with the administration of radiotherapy to a subject in accordance with an illustrative embodiment.

FIG. 10 depicts an example screenshot of a user interface for presenting summarized records associated with the administration of radiotherapy to a subject in accordance with an illustrative embodiment.

FIG. 11 depicts an example screenshot of a user interface for presenting notifications associated with the administration of radiotherapy to a subject in accordance with an illustrative embodiment.

FIG. 12 depicts a flow diagram of a method of managing radiotherapy informatics for subjects using data of disparate modalities in accordance with an illustrative embodiment.

FIG. 13 depicts a block diagram of a server system and a client computer system, in accordance with one or more implementations.

### DETAILED DESCRIPTION

Following below are more detailed descriptions of various concepts related to, and embodiments of, systems and methods for managing radiotherapy informatics for subjects using data of disparate modalities. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes systems and methods for managing radiotherapy informatics for subjects using data of disparate modalities.

Section B describes a network environment and computing environment which may be useful for practicing various computing related embodiments described herein.

### A. Systems and Methods for Managing Radiotherapy Informatics for Subjects Using Data of Disparate Modalities

Radiation Therapy (RT), a treatment for cancer, employs high doses of radiation to accurately eliminate cancer cells and reduce tumor size. For over two-thirds of cancer patients, RT forms a crucial part of their treatment regimen. The process unfolds from initial patient consultation through various stages, including simulation, contouring, treatment planning, plan verification, and ultimately, the administration of therapy. This sequence utilizes advanced algorithms, precision equipment, and stringent quality controls to direct radiation to the patient. Central to the effectiveness of RT is the patient's digital profile, which compiles comprehensive patient data from various sources such as radiology images, pathology slides, medical reports, and RT diagrams and plans, alongside demographic, medical history, medication, genomic, and social information. This profile is dynamic, providing real-time updates on the patient's health status. Designed with precision, RT aims to target cancer cells while sparing surrounding healthy tissue, minimizing radiation-related side effects. Achieving this requires a coordinated effort from a multidisciplinary team including radiation oncologists, medical physicists, dosimetrists, radiation therapists, computer scientists, and nurses. This collaboration hinges on the meticulous sharing and exchange of the patient's digital profile, allowing for synchronized operations, seamless workflow, and effective communication throughout the treatment process.

One significant hurdle in radiotherapy is the dispersion of patient data across varied locations, systems, and modalities, a situation especially prevalent in large healthcare organizations. This dispersion often necessitates manually assembling the patient's digital profile by navigating through diverse systems, dealing with multiple vendors, and converting data from various formats, a process that is both time-consuming and prone to errors.

Furthermore, simply centralizing patient data does not automatically result in the creation of a functional digital profile. The true value of this data emerges when it is interconnected, reflecting the patient's health status in real-time. Given the fluid nature of a patient's health before, during, and after treatment, the digital profile must be continuously updated to accurately reflect current conditions. The challenge extends to converting and standardizing patient data into universally accepted formats for use throughout the radiotherapy process.

Effective radiotherapy treatment relies on a structured care team, where seamless communication, coordination, and access to the patient's digital profile are essential for success and error prevention. Real-time updates to the patient's status within this profile are crucial for the team to swiftly adapt to any changes during treatment. However, reliance on email for communication complicates tracking patient updates among team members, particularly in high-volume settings, leading to communication fatigue and potential treatment risks. These major challenges, combined with the dynamic nature of clinical protocols and the shifting of treatment schedules and methods, often result in a delay of two weeks or more before the commencement of the first radiotherapy session. This delay is critical, as timely treatment can curb tumor growth and alleviate psychological distress for cancer patients.

To address these and other technical challenges, presented herein is a radiotherapy informatics platform. This platform addresses the prevalent challenges faced in daily radiotherapy (RT) clinics by facilitating the harmonization and integration of patient data into comprehensive digital profiles. A unique connector layer may be designed for flexibility, enabling seamless integration with all major vendor systems. Utilizing leading distributed big data technologies, connectivity with over hospital-wide systems, including common RT vendor systems, can be achieved. This integration spans multiple data modalities, from pathology and radiology reports to diagnostic and simulation digital imaging and communications in medicine (DICOM) images and daily cone beam computed tomography (CT) scans, forming the cornerstone of the patient digital profile. The platform processes data in stages, from nightly batches that consolidate daily updates to real-time ingestion capturing immediate changes and clinical events, providing medical professionals with access to up-to-date and accurate patient profiles from any location and device.

The automated curation engine of the platform, leveraging the latest medical large language models (LLMs) and knowledge graphs, can transform this data into actionable insights. This includes patient matching, data standardization, and the extraction of structured information from free-text reports, all in alignment with standards (e.g., set by the American Association of Physicists in Medicine (AAPM) and Fast Healthcare Interoperability Resources (FHIR)). An assistant feature, guided by human expertise, ensures the alignment of curation processes, filling gaps in patient data to maintain high-quality digital profiles.

Addressing communication challenges within the RT care team, the platform serves as a centralized communication hub, streamlining interactions and reducing the reliance on inefficient email exchanges. With features designed for real-time notifications, discussion threads based on treatment steps, and integration with various messaging platforms, it facilitates clear, concise communication across the care team. A visually intuitive “subway” map tracks the progress of each treatment step, providing a comprehensive overview of clinic performance and enhancing treatment planning clarity. The platform can incorporate automatic checks and AI-driven algorithms for contour quality assessment and smart scheduling. By monitoring the entire dose delivery process and employing over-automated checkers for plan quality and dosage constraints, adherence may be checked. Moreover, the platform can be used as a research tool, enabling the construction of patient cohorts through big data integration and standardized data. With an integrated API for data accessibility, users (e.g., researchers) can develop and test models using real-world data directly within the clinical workflow, bridging the gap between research and practical application in daily RT clinics.

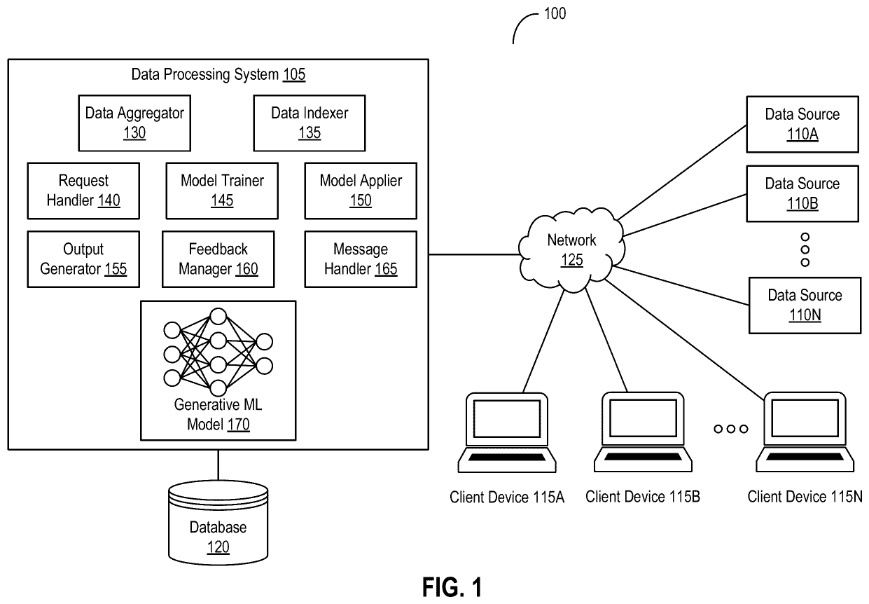

Referring now to FIG. 1, depicted is a block diagram of a system 100 for managing radiotherapy informatics for subjects using data of disparate modalities. The system 100 may include at least one data processing system 105, a set of data sources 110A-N (hereinafter generally referred to as data sources 110), a set of client device 115A-N (hereinafter referred to as client devices 115), and at least one database 120, among others, coupled with one another via at least one network 125. The data processing system 100 may include at least one data aggregator 130, at least one data indexer 135, at least one request handler 140, at least one model trainer 145, at least one model applier 150, at least one output generator 155, at least one feedback manager 160, at least one message handler 165, and at least one generative machine learning (ML) model 170, among others. Each of the components in the system 100 (such as the data processing system 105 and its subcomponents, data sources 110, and the client device 115) as detailed herein may be implemented using hardware (e.g., one or more processors coupled with memory), or a combination of hardware and software as detailed herein in Section B.

In further detail, the data processing system 105 may be any computing device including one or more processors coupled with memory and software and capable of performing the various processes and tasks described herein. The data processing system 105 may be associated with a platform for providing a portal to manage administration of radiotherapy to subjects. The data processing system 105 may be in communication with the data sources 110, the client device 115, and the database 120, among others, via the network 125. The data processing system 105 may be situated, located, or otherwise associated with at least one server group. The server group may correspond to a data center, a branch office, or a site at which one or more servers corresponding to the data processing system 105 is situated.

The data processing system 105 may include one or more modules, components, or subsystems to perform the various processes and tasks described herein. The data aggregator 130 may retrieve data associated with subjects from the set of data sources 110 over the network 125. The data indexer 135 may convert the retrieved data into structured data according to a format for storage and maintenance on the database 120. The request handler 140 may receive and process queries regarding data on the database from the client device 115. The model trainer 145 may initialize, train, and establish the generative ML model 170 using training data. The model applier 150 may apply input prompts created from queries to the generative ML model 170 to produce output. The output generator 155 may provide information generated using the output from the generative ML model 170. The feedback manager 160 may receive and process responses from the client device 115. The message handler 165 may facilitate communication of messages among the data sources 110 and the client devices 115.

The generative ML model 170 may include any network architecture to generate output content (e.g., radiotherapy timeline) using an input prompt (e.g., information for a given subject). For example, the generative ML model 170 may generate a predicted tokens (e.g., portions of text or images) from the input tokens (e.g., portions of images or words) of the prompt. The network architecture for the generative ML model 170 may generally be a deep learning architecture, such as a transformer model (e.g., a generative pre-trained transformer (GPT), a bidirectional encoder representation transformers (BERT), or a text-to-text transfer transformer (T5)), or a recurrent neural network (RNN), among others. In some embodiments, the input and output may be of the same modality (e.g., text-to-text, audio-to-audio, or image-to-image). In some embodiments, the input and output may be of different modalities (e.g., text to audio, text to image, audio to text, audio to image, image to audio, or image to text). While the generative ML model 170 is primarily described herein in terms of text inputs and outputs of text, audio or image, any combination of modalities may be processed and generated by the generative ML model 170. In some embodiments, the generative ML model 170 may be maintained on or by the data processing system 105. In some embodiments, the generative ML model 170 may be maintained on a separate service in communication with the data processing system 105. In some embodiments, the generative ML model 170 may include a summarizer (e.g., based on the transformer model or natural language processing (NLP) algorithms).

In some embodiments, the generative ML model 170 can include a set of weights arranged across a set of layers in accordance with the transformer architecture. Under the architecture, the generative ML model 170 can include at least one tokenization layer, at least one input embedding layer, at least one position encoder, at least one encoder stack, at least one decoder stack, and at least one output layer, among others, interconnected with one another (e.g., via forward, backward, or skip connections). The tokenization layer can convert input in the form of a set of strings (or data in other modalities) into a corresponding set of word vectors (also referred to herein as tokens or vectors) in an n-dimensional feature space. The position encoder can generate positional encodings for each input embedding as a function of a position of the corresponding word vector or by extension the string within the input set of strings.

Continuing on, the encoder stack can include a set of encoders, each including at least one attention layer and at least one feed-forward layer, among others. The attention layer (e.g., a multi-head self-attention layer) can calculate an attention score for each input embedding to indicate a degree of attention the embedding is to place focus on and generate a weighted sum of the set of input embeddings. The feed-forward layer can apply a linear transformation with a non-linear activation (e.g., a rectified linear unit (ReLU)) to the output of the attention layer. The output can be fed into another encoder in the encoder stack in the transformer layer.

In addition, the decoder stack can include at least one attention layer, at least one encoder-decoder attention layer, and at least one feed-forward layer, among others. In the decoder stack, the attention layer (e.g., a multi-head self-attention layer) can calculate an attention score for each output embedding (e.g., embeddings generated from a target or expected output). The encoder-decoder attention layer can combine inputs from the attention layer in the decoder stack and the output from one of the encoders in the encoder stack and can calculate an attention score from the combined input. The feed-forward layer can apply a linear transformation with a non-linear activation (e.g., a rectified linear unit (ReLU)) to the output of the encoder-decoder attention layer.

The output layer of the generative ML model 170 can include at least one linear layer and at least one activation layer, among others. The linear layer can be a fully connected layer to perform a linear transformation on the output from the decoder stack to calculate token scores. The activation layer can apply an activation function (e.g., a softmax, sigmoid, or rectified linear unit) to the output of the linear function to convert the token scores into probabilities (or distributions). The probability may represent a likelihood of occurrence for an output token, given an input token. The output layer can use the probabilities to select an output token (e.g., at least a portion of output text, image, audio, video, or multimedia content with the highest probability). Repeating this over the set of input tokens, the resultant set of output tokens can be used to form the overall output of the generative ML model 170. While described primarily herein in terms of transformer models, the data processing system 105 can use other model architectures to generate and output content.

The set of data sources 110 may include any number of devices in communication with the data processing system 105 to communicate data associated with subjects under evaluation. Each data source 110 may be associated with an entity examining or evaluating the subject, such as a hospital, a clinical laboratory, an imaging center, a radiology department, a pharmacy, or a vendor, among others. The data source 110 may be in communication with the data processing system 105, the client device 115, and the database 120, via the network 125. In some embodiments, the data source 110 may be any computing device comprising one or more processors coupled with memory and software and capable of performing the various processes and tasks described herein. The data source 110 may retrieve, obtain, or otherwise receive data about the subject under evaluation, such as pathology reports, radiology reports, or information on clinically relevant events, among others. In some embodiments, at least one data source 110 may correspond to at least one client device 115, or vice-versa.

In some embodiments, the data source 110 may be an imaging device to acquire biomedical image of the subject. The biomedical image may be acquired in any number of imaging modalities, such as a whole slide imaging (WSI) modality, a computed tomography (CT) modality, a magnetic resonance imaging (MRI) modality, a positron emission tomography (PET) modality, or an x-ray imaging modality, among others. The biomedical image may be of an organ under evaluation for cancer in the subject. The data or biomedical images may be maintained in any number of formats, such as Digital Imaging and Communications in Medicine (DICOM), Health Level 7 (HL7), a portable document format (PDF), extensible markup language (XML), comma-separated values (CSV), or JavaScript Object Notation (JSON), among others.

Each client device 115 (sometimes herein referred to as a user computing device) may be any computing device comprising of one or more processors coupled with memory and software and capable of performing the various processes and tasks described herein. The client device 115 may be in communication with the data processing system 105, the data sources 110, and the database 120 via the network 125. The client device 115 may have at least one display. The client device 115 may be associated with an entity (e.g., a radiotherapy planner, a simulation planner, radiotherapist, oncologist, physicist, or clinician) involved in provision of radiotherapy to a subject at risk of or diagnosed with cancer. The display may present information about the subject provided by the data processing system 105. The client device 115 may also be in communication with a radiotherapy device used to emit, deliver, or otherwise administer the radiotherapy to a subject. The radiotherapy device may include, for example, a linear accelerator (LINAC), a gamma knife, a robotic radiosurgery device, or a brachytherapy applicator, among others.

The database 120 (sometimes herein referred to as a data repository) may store and maintain various resources and data associated with the data processing system 105, the data sources 110, and the client device 115, among others. The database 120 may include a database management system (DBMS) to arrange and organize the data maintained thereon. The database 120 may be in communication with the data processing system 105, the data sources 110, and the client device 115 via the network 125. While running various operations, the data processing system 105, the data source 110, and the client device 115 may access the database 120 to retrieve identified data therefrom. The data processing system 105, the data source 110, and the client device 115 may also write data onto the database 120 from running such operations.

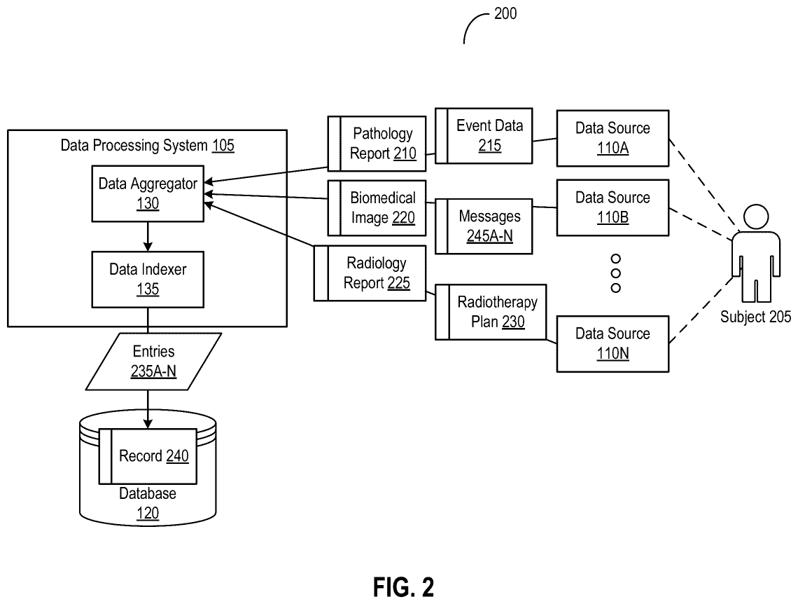

Referring now to FIG. 2, among others, depicted a block diagram of a process 200 to collect data in various modalities in the system 100 for managing radiotherapy informatics. The process 200 may include or correspond to operations performed in the system 100 to maintain profiles for subjects using data associated with the subjects retrieved from various data sources. Under the process 200, the data aggregator 130 executing on the data processing system 105 may retrieve, identify, or otherwise receive data associated with at least subject 205 from one or more of the data sources 110. The data may be received by the data aggregator 130 in accordance with a schedule. For instance, the data aggregator 130 may invoke a given data source 110 via an application programming interface (API) to retrieve the data from the data source 110. In some embodiments, the data aggregator 130 may monitor for events associated with the subject 205 in a network environment including the data sources 110 and the client devices 115. For example, the data aggregator 130 may use one or more APIs (e.g., representational state transfer (REST) API, a fast healthcare interoperability resources (FHIR) by HL7 message protocol, EPIC electronic health record (EHR) and electronic medical record (EMR) protocols) to monitor for data associated with events. The data may be received by the data aggregator 130 in real-time or near-real-time (e.g., within 10 seconds to 1 day).

The subject 205 may be a human or animal subject, among others. The subject 205 may be at risk of, diagnosed with, or otherwise under evaluation for cancer. The cancer may include, for example, lung cancer, brain cancer, head and neck cancer, colon cancer, rectal cancer, uterine cancer, endometrial cancer, stomach cancer, prostate cancer, ovarian cancer, cervical cancer, bladder cancer, or breast cancer, among others. The cancer may be associated with an organ in the subject 205. The organ may correspond to the one at risk of, diagnosed with, or under evaluation for the cancer. The organ may include, for example, lung, brain, head, neck, colon, rectum, uterus, endometrium, stomach, ovary, cervix, bladder, prostate, or breast, among others. The subject 205 may have been administered with a radiotherapy to treat the cancer or may be under evaluation for planning of the radiotherapy to treat the cancer. The radiotherapy can include, for example intensity-modulated radiation therapy (IMRT), an external beam radiotherapy (EBRT), stereotactic body radiation therapy (SBRT), image-guided radiation therapy (IGRT), or brachytherapy, among others.

The data received by the data aggregator 130 may be in different modalities (e.g., text or visual). The data may correspond to events occurring in the network environment including the data sources 110 and the client devices 115. The data may include, for example, at least one pathology report 210, at least one event data 215, at least one biomedical image 220, or at least one radiology report 225, among others. The events may correspond to any occurrences of detected actions in the network environment associated with the subject 205 to be provided with radiotherapy. The events may include, for example, approval of radiotherapy, generation of radiotherapy simulation, provision of radiotherapy, subject diagnosis, or an acquisition of a biomedical image, among others. In some embodiments, the data aggregator 130 may retrieve, identify, or otherwise receive data defining the pathology report 210 from one or more of the data sources 110. At least one of the data sources 110 may produce, create, or otherwise generate the pathology report 210. The pathology report 210 may be an electronic document containing the findings from a pathology test on a biological sample (e.g., a tissue sample) from an organ of the subject 205 associated with the cancer. The pathology report 210 may identify or include a set of characteristics defining the cancer in the organ of the subject 205. The set of characteristics may identify or include, for example, one or more of: a cancer type (e.g., identification of cancer), a tumor classification (e.g., staging information), a tumor size (e.g., physical size), a tumor appearance (e.g., gross description), or a tumor grade (e.g., degree of abnormality), among others. The pathology report 210 may also include or identify one or more of, for example, subject information (e.g., name, age, gender, or record number), clinical history, pathologist information (e.g., name or institute), test result, or final diagnosis, among others.

The data aggregator 130 may retrieve, identify, or otherwise receive data defining the event data 215 from one or more of the data sources 110. At least one of the data sources 110 may produce, create, or otherwise generate the event data 215 associated with the subject 205. The event data 215 may include information on events occurring with respect to the subject 205, such as those events affecting the health of the subject 205 or previous or scheduled delivery of treatment (e.g., radiotherapy for the cancer), among others. The event data 215 may be generated by a clinician examining the subject 205, for example, by inputting the information for the event data 215 on a computing device (e.g., corresponding the data source 110) at the hospital to record the events. The event data 215 may identify or include a set of event identifier for a corresponding set of events associated with the subject 205. The event data 215 may identify or include a set of timestamps (e.g., include date and time) for the corresponding set of events. The events associated with the subject 205 may include, for example, one or more of: approval of radiotherapy, generation of radiotherapy simulation, administration of radiotherapy (e.g., in the past), scheduled administration of radiotherapy (e.g., in the future), subject diagnosis, report of effects from the administration of radiotherapy, report of symptoms, acquisition of biomedical image, biopsy of cancer, resection of tumors associated with cancer, prescription of drugs for the cancer, taking of drugs for cancer, or acquisition of biomedical images, among others. The event data 215 may identify or include at least one identifier associated with a clinician (e.g., an examining physician, radiologist, pathologist, or nurse) for at least one of the events.

The data aggregator 130 may retrieve, identify, or otherwise receive data defining the biomedical image 220 from one or more of the data sources 110. In some embodiments, the data aggregator 130 may receive the data defining a set of biomedical images 220. At least one of the data sources 110 may produce, create, or otherwise generate the biomedical image 220. The biomedical image 220 may be of the organ in the subject 205 associated with the cancer. For instance, the biomedical image 220 may correspond to a two-dimensional slice or a three-dimensional volume containing at least a portion (e.g., a tumorous portion or a tissue to be evaluated) of the organ associated with the cancer in the subject 205. The biomedical image 220 may be acquired by an imaging device for the given imaging modality. The biomedical image 220 may be acquired in accordance with any imaging modality, such as a whole slide imaging (WSI) modality, a computed tomography (CT) modality, a magnetic resonance imaging (MRI) modality, a positron emission tomography (PET) modality, or an x-ray imaging modality, among others. When multiple biomedical images 220 are provided, the biomedical images 220 may be in one or more imaging modalities.

The data aggregator 130 may retrieve, identify, or otherwise receive data defining the radiology report 225 from one or more of the data sources 110. At least one of the data sources 110 may produce, create, or otherwise generate the radiology report 225. The radiology report 225 may be associated with the biomedical image 220 acquired in accordance with a tomographic imaging modality, such as CT, MRI, PET, or x-ray, among others. The radiology report 225 may include information providing a description of the biomedical image 220. The radiology report 225 may identify or include a set of characteristics for the biomedical image 220, such as imaging modality, field of view, anatomical location, and findings (e.g., diagnosis of cancer), among others.

The data aggregator 130 may retrieve, identify, or otherwise receive data defining a simulated radiotherapy plan 230 from one or more of the data sources 110. At least one of the data sources 110 may produce, create, or otherwise generate the simulated radiotherapy plan.

The sample subject may have been evaluated for treatment of cancer and may be under evaluation to determine whether administration of the radiotherapy will be performed to treat the cancer. The simulated radiotherapy plan 230 may be generated by a clinician (e.g., radiology) using radiotherapy simulation software on the data source 110. The simulated radiotherapy plan may identify or include a set of parameters defining the simulated radiotherapy under to the subject 205 at a given time. The set of parameters may identify or include, for example, one or more of: a target volume (e.g., three-dimensional space encompassing the tumor for delivery of radiotherapy), a dose prescription (e.g., total amount of radiation delivered to the target volume), a dose distribution (e.g., spatial arrangement of radiation in the target volume), a beam configuration (e.g., angle, energy, shape, and other arrangement of radiation beam), or a radiotherapy type (e.g., IMRT, IGRT, EBRT, SBRT, or brachytherapy), among others.

The data aggregator 130 may retrieve, identify, or otherwise receive data defining a radiotherapy plan 230 from one or more of the data sources 110. At least one of the data sources 110 may produce, create, or otherwise generate the previous radiotherapy plan 230. The radiotherapy plan 230 may be generated by a clinician (e.g., radiologist) that administered the radiotherapy to the subject 205 by inputting the information on a computing device (an example of the data source 110). The radiotherapy plan 230 may identify or include a set of parameters defining the radiotherapy administered to the subject 205 at a defined prior time. The set of parameters may identify or include, for example, one or more of: a target volume, a dose prescription, a dose distribution, a beam configuration, or a radiotherapy type, among others.

Although the data (e.g., the pathology report 210, the event data 215, the biomedical image 220, the radiology report 225, and the radiotherapy plan 230) are depicted as received from different data sources 110, the data aggregator 130 may receive the data from at least one of the data sources 110.

The data aggregator 130 may collect, obtain, or otherwise receive one or more messages 245A-N (hereinafter generally referred to as messages 245) from one or more of the data sources 110 (or the one or more of the client devices 115). The messages 245 may be communicated between devices (e.g., data sources 110 or client devices 115) of users (e.g., radiotherapy planner, a simulation planner, radiotherapist, oncologist, physicist, or clinician) involved in the administration of radiotherapy to the subject 205. Each message 245 may contain or include content, such as a set of alphanumeric characters, image, video, or audio (e.g., inputted by the sending user) and metadata, such as sender identifier, a recipient identifier, and a timestamp identifying a time at which the message 245 is sent, among others. The message 245 can correspond to or include, for example, a short message service (SMS) message, a multimedia messaging service (MMS), an electronic mail, or an invocation of an API of a messaging protocol to communicate among the devices. When the message 245 is an electronic mail, the sender and recipient identifiers may include a mail server address corresponding to the data processing system 105. The data processing system 105 may use the address to route the message 245 to the identified recipient client device 115.

The data indexer 135 executing on the data processing system 105 may store and maintain at least one record 240 for the subject 205. The record 240 may be maintained using the data received from the one or more of the data sources 110. The record 240 may identify or include one or more of: the pathology report 210, the event data 215, the radiology report 225, the biomedical image 220, simulated radiotherapy plan 230, or previous radiotherapy plan, among others. With detection of the event in the network environment, the data indexer 135 may modify or update the record 240 using the newly received data associated with the event. In some embodiments, the record 240 may identify or include the pathology report 210, including the set of characteristics defining the cancer in the organ of the subject 205 and the biomedical image 220 of the organ in the subject 205. In some embodiments, the record 240 may also identify or include the radiology report 225, the simulated radiotherapy plan 230, or the previous radiotherapy plan, or the event data 215 for the event associated with the subject 205. The record 240 may be stored and maintained by the data indexer 135 on the database 120 using one or more files, such as Digital Imaging and Communications in Medicine (DICOM), Health Level 7 (HL7), a portable document format (PDF), extensible markup language (XML), or comma-separated values (CSV), among others.

In maintaining the record 240, the data indexer 135 may create, write, or otherwise generate a set of entries 235A-N (hereinafter generally referred to as entries 235) in accordance with at least one template based on the received data. The data for the events received from the one or more of the data sources 110 may be in different formats or standards or may be unstructured data (e.g., with the pathology report 210, the event data 215, the radiology report 225, the radiotherapy plan 230, the messages 245, or data derived therefrom). The template may specify or define conversion of the received original data in the initial format to the entries 235. For instance, the template may specify a set of field-value pairs to be included in the entry 235 for the pathology report 210, the event data 215, the radiology report 225, the radiotherapy plan 230, or the messages 245. The data indexer 135 may transform or convert the received data into the entries 235 using the template. To convert, the data indexer 135 may process or parse the data to identify information associated with a given field defined in the template. With the identification, the data indexer 135 may extract or identify the information and include the information as the value in the entry 235 in accordance with the template. The data indexer 135 may insert, add, or otherwise include the set of entries 235 as part of the record 240 for the subject 205. As additional data are received from the data sources 110, the data indexer 135 may update the record 240 using the additionally received data.

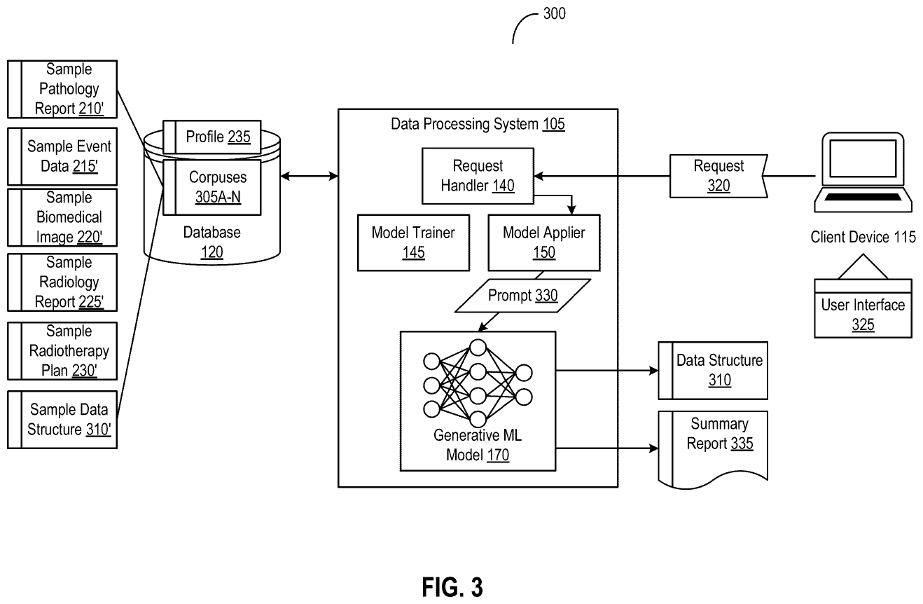

FIG. 3 depicts a block diagram of a process 300 to apply generative machine learning (ML) models to the data in the system 100 for managing radiotherapy informatics. The process 300 may include or correspond to operations performed in the system 100 in using generative ML models to create output data for radiotherapy plans. For the process 300, the model trainer 145 executing on the data processing system 105 may initialize, train, and establish the generative ML model 170. The model trainer 145 may initialize the generative ML model 170 by assigning or setting values (e.g., random values) to the set of weights of generative ML model 170. To train, the model trainer 145 may retrieve, identify, or otherwise identify a set of corpuses 305A-N (hereinafter generally referred to as corpuses 305). In some embodiments, the model trainer 145 may use a pre-trained model as the generative ML model 170 and use the set of corpuses 305 to fine-tune the generative ML model 170. The set of corpuses 305 may be stored and maintained on the database 120 (e.g., as depicted) or another data storage. Each corpus 305 may include data to be used to train the generative ML model 170. In some embodiments, at least one corpus 305 may be a generalized dataset (e.g., without any focus to a particular knowledge domain).

At least one corpus 305 may include a knowledge-domain specific dataset. The corpus 305 may identify or include at least one sample pathology report 210′, at least one sample event data 215′, at least one sample radiology report 225′, at least one sample biomedical image 220′, sample radiotherapy plan 230′, at least one sample data structure 310′, or one or more messages, among others. The corpus 305 may correspond to data previously generated for a given sample subject at risk of or diagnosed with cancer undergoing administration of radiotherapy. The pathology report 210′ may be similar to the pathology report 210. The sample pathology report 210 may identify or include a set of characteristics defining a cancer in an organ in a sample subject. The sample event data 215′ may be similar to the event data 215. The sample event data 215′ may identify or include a set of event identifiers and a set of timestamps (e.g., include date and time) for a corresponding set of events associated with the sample subject. The sample biomedical image 220′ may be similar to the biomedical image 220. The sample biomedical image 220′ may be of the organ in the sample subject associated with the cancer and may be in accordance with any imaging modality.

In addition, the sample radiology report 225′ may be similar to the radiology report 225. The sample radiology report 225′ may be associated with the sample biomedical image 220′ (e.g., acquired via a tomographic imaging modality), and may include a set of characteristics for the sample biomedical image 220′. The sample radiotherapy plan 230′ may define or characterize a previous administration of a radiotherapy to the organ of the sample subject associated with the cancer. The sample radiotherapy plan 230′ may be similar to the radiotherapy plan 230. The sample subject may have been administered with the radiotherapy for the cancer and may be under evaluation to determine whether additional administration of the radiotherapy is to be performed to treat the cancer. The radiotherapy plan 230 may be generated by a clinician (e.g., radiology) that administered the radiotherapy to the subject 205 by inputting the information on a computing device (an example of the data source 110). The messages in the corpus 305 may be similar to the messages 245. The messages may be communicated between devices (e.g., data sources 110 or client devices 115) of users (e.g., radiotherapy planner, a simulation planner, radiotherapist, oncologist, physicist, or clinician) in a previous administration of a radiotherapy to the subject.

In the corpus, the sample data structure 310′ may define or include informatics to present for the associated sample pathology report 210′, the sample event data 215′, the sample biomedical image 220′, the sample radiology report 225′, or the sample radiotherapy plan 230′, the messages, or any combination thereof. The presentation of the informatics may be defined using one or more files, such as JavaScript, HyperText Markup (HTML), or Extendible Markup (XML), among others. In some embodiments, the sample data structure 310′ may include or identify a set of nodes and a set of edges, among others. Each node may correspond to a respective event in the sample event data 215′. Each node may correspond to a user interface element (e.g., a button or icon) to provide information (e.g., a summary of the sample pathology report 210′, a summary of the sample radiology report 225′, a summary of the sample radiotherapy plan 230′, the sample biomedical image 220′, a summary of one or more messages, or a descriptor of the event) on the associated with upon interaction. In addition, each edge may define a relationship between a corresponding pair of nodes corresponding to at least a pair of events. The relationship may include a chronological relationship (e.g., indicating that one event corresponding to one node occurs prior to a subsequent event for another node) and a dependency relationship (e.g., indicating that one event corresponding to one node is a condition precedent to a subsequent event for another node), among others. For example, the set of nodes and edges in the sample data structure 310′ may be arranged sequentially in accordance with the set of timestamps in the event data 215′.

With the identification of each corpus 305, the model trainer 145 may identify or select a portion of the corpus 305 as a source dataset (e.g., the sample pathology report 210′, the sample event data 215′, the sample biomedical image 220′, the sample radiology report 225′, or the sample radiotherapy plan 230′) and another portion of the corpus 305 as a destination dataset (e.g., the sample data structure 310′). The source set may be used as input into the generative ML model 170 to produce an output to be compared against the destination set. The model trainer 145 may feed or apply the source set as input into the generative ML model 170. In applying, the model trainer 145 may process the source set in accordance with the set of weights of the generative ML model 170. The model trainer 145 may produce or generate an output from applying the input source set to the generative ML model 170. The output may be comprised of a set of tokens (or a set of subcomponents forming the overall output).

Upon generation, the model trainer 145 may compare the output from the generative ML model 170 with the destination set. The comparison may be between a distribution (or probabilities) of the tokens in the output versus a distribution (or probabilities) of the tokens in the destination set). Based on the comparison, the model trainer 145 may calculate, determine, or generate at least one loss metric. The loss metric may indicate a degree of deviation of the output from the expected output as defined by the target set of the corpus 305.

The loss metric may be calculated in accordance with any number of loss functions, such as a norm loss (e.g., L1 or L2), mean squared error (MSE), quadratic loss, cross-entropy loss, or Huber loss, among others. In some embodiments, the model trainer 145 may generate at least one similarity metric based on the comparison. The degree of similarity may be in accordance with any number of similarity measures, such as cosine similarity, Jaro distance, Jaccard index, or Dice coefficient, among others.

Using the loss metric, the model trainer 145 can update one or more weights in the set of layers of the generative ML model 170. The updating of the weights may be in accordance with a back propagation and optimization function (sometimes referred to herein as an objective function) with one or more parameters (e.g., learning rate, momentum, weight decay, and number of iterations). The optimization function may define one or more parameters at which the weights of the generative ML model 170 are to be updated. The optimization function may be in accordance with stochastic gradient descent, and may include, for example, an adaptive moment estimation (Adam), implicit update (ISGD), and adaptive gradient algorithm (AdaGrad), among others. The model trainer 145 can iteratively train the generative ML model 170 until convergence. Upon convergence, the model trainer 145 can store and maintain the set of weights for the set of layers of the generative ML model 170 for use in inference stage.

With the establishment of the generative ML model 170, the request handler 140 executing on the data processing system 105 may retrieve, identify, or otherwise receive at least one request 320 from the client device 115. The request 320 may be for retrieval of radiotherapy informatics for the subject 205. The request 320 may also include an identifier for the subject 205 (e.g., using an anonymized identifier) or an identifier for the record 240 associated with the subject 205. The request 320 may be generated and transmitted in response to user interactions on the user interface 325 presented on the client device 115. In some embodiments, the request 320 may be to generate analytics information for the subject 205. In some embodiments, the request 320 may be to retrieve clinician assignment availability for the subject 205. In some embodiments, the request 320 may be to summarize the record 240 associated with the subject 205. The request 320 may identify a user type of the client device 115. The user type may include, for example, radiotherapy planner, a simulation planner, radiotherapist, oncologist, physicist, or clinician, among others. The request 320 may indicate that any one or more of the event data 215, reports (e.g., the pathology report 210 or the radiology report 225), radiotherapy plan 230, or messages 245 are to be summarized.

In some embodiments, the request handler 140 may send, transmit, or otherwise provide instructions for at least one user interface 325 to the client device 115. The instructions may identify or define rendering and operations of the user interface 325. The user interface 325 may be a graphical user interface (GUI) including a set of user interface elements. The user interface elements may be used to define or generate the request 320. In some embodiments, the user interface 325 may be part of a web application to be loaded by an application (e.g., a web browser) or a separate, dedicated application on the client device 115. Using the user interface 325, the user of the client device 115 (e.g., a clinician examining the subject 205) may interact with the user interface elements to define the request 320. Using the inputs on the user interface 325, the client device 115 may generate the request 320 and may send, transmit, or otherwise provide the request 320 to the data processing system 105.

The request handler 140 may create, write, or otherwise generate at least one prompt 330 based on at least a portion of the record 240. The prompt 330 may be generated in response to a detection of one or more events in the network environment. In some embodiments, the request handler 140 may generate the prompt 330 based on the request 320 and at least the portion of the record 240 (e.g., the messages or reports). The prompt 330 may be generated in response to receipt of the request 320. The prompt 330 may contain or include information from the request 320 and the record 240 to be inputted to the generative ML model 170. From the database 120, the request handler 140 may retrieve, select, or identify the record 240 associated with the subject 205 as identified in the request 320. The request handler 140 may use information from the record 240 to generate the prompt 330. In addition, the prompt 330 may identify or include one or more of: the pathology report 210, the event data 215, the biomedical image 220, the radiology report 225, simulated radiotherapy plan 230, or previous radiotherapy plan, among others.

In generating, the request handler 140 may use a template defining the generation of the prompt 330. The template may include placeholders for information to be obtained from the request 320 or the record 240. Each placeholder may specify or define a field from the request 320 or the entries 235 of the record 240 to the prompt 330. For instance, the prompt 330 created using the template may include the text “Please create a radiotherapy track line for [subject identifier] based on the following information” as defined by the request 320 and information from the pathology report 210 and the biomedical image 220. The request handler 140 may add or include the information from the request 320 and the record 240 into the template. In some embodiments, when the request 320 is for summarization of the record 240, the request handler 140 may identify or select the template for the user type identified in the request 320. The templates may be particularized for the role indicated by the user type. For instance, when the user type is oncologist, the template may indicate that comprehensive patient history, prior therapies, key imaging, or pathology data, among others, are to be summarized. When the user type indicates physicist, the template may identify dose delivery details, QA data, and technical machine parameters, among others, are to be summarized. When the user type indicates planner or radiotherapist, the prompt may indicate that scheduling and safety checkpoints, among others, are to be included. With the identification of the template for the user type, the request handler 140 may generate the prompt 330.

The model applier 150 executing on the data processing system 105 may feed or apply the prompt 330 to the generative ML model 170. In applying, the model applier 150 may process the prompt 330 in accordance with the set of weights of the generative ML model 170. Based on applying the prompt 330 to the generative ML model 170, the model applier 150 may produce or generate the data structure 310. The data structure 310 may be similar in form to the sample data structure 310'. The data structure 310 may define or include informatics to present for the associated pathology report 210, the event data 215, the biomedical image 220, the radiology report 225, the radiotherapy plan 230, and other information from the record 240, or any combination thereof. The presentation of the informatics may be defined using one or more files, such as JavaScript, HyperText Markup (HTML), or Extendible Markup (XML), among others.

In some embodiments, the data structure 310 (e.g., to define a timeline) may include or identify a set of nodes and a set of edges, among others. Each node may correspond to a respective event. Each node may correspond to a user interface element (e.g., a button or icon) to provide information (e.g., a summary of the pathology report 210, a summary of the radiology report 225, a summary of the radiotherapy plan 230, the biomedical image 220, or a descriptor of the event) on the associated with upon interaction. In addition, each edge may define a relationship between a corresponding pair of nodes corresponding to at least a pair of events. The relationship may include a chronological relationship (e.g., indicating that one event corresponding to one node occurs prior to a subsequent event for another node) and a dependency relationship (e.g., indicating that one event corresponding to one node is a condition precedent to a subsequent event for another node), among others. For example, the set of nodes and edges in the data structure 310 may be arranged sequentially, in accordance with the set of timestamps in the event data 215.

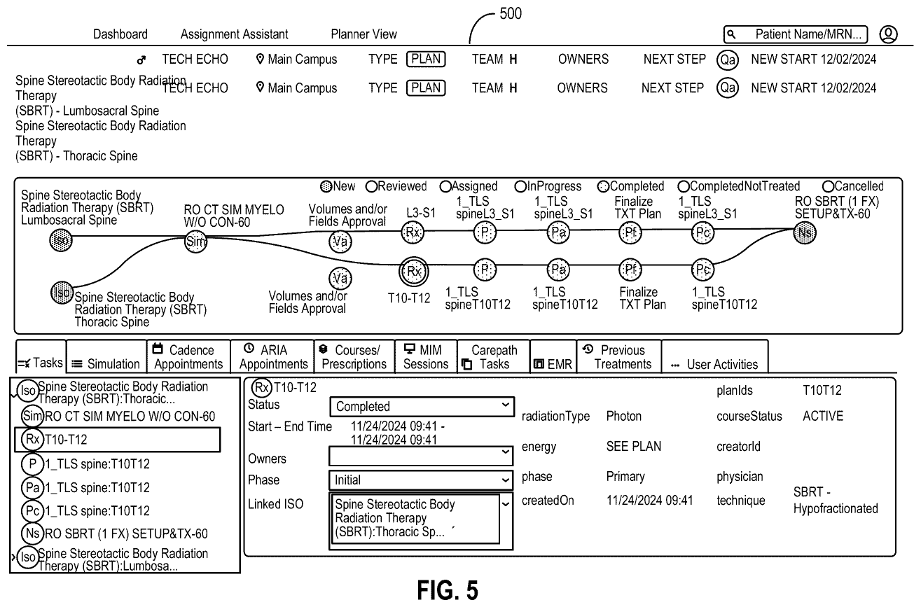

The data structure 310 may include a graphical representation of the information for events (e.g., as identified in the event data 215) occurring in conjunction with the administration of the radiotherapy to the subject 205. The data structure 310 may include the set of event identifiers and the include a set of timestamps (e.g., include date and time) for the corresponding set of events. The events associated with the subject 205 may include, for example, one or more of: approval of radiotherapy, generation of radiotherapy simulation, administration of radiotherapy, subject diagnosis, report of effects from the administration of radiotherapy, report of symptoms, acquisition of biomedical image, biopsy of cancer, or resection of tumors associated with cancer, among others. An example of the timeline may be illustrated in FIG. 5.

In some embodiments, the model applier 150 may generate a set of candidate radiotherapy plans for at least one of the events based on the application of the prompt 330 to the generative ML model 170. The radiotherapy plan may identify or include a set of parameters defining the radiotherapy to be administered to the subject 205 at the defined time corresponding to the event. The set of parameters may identify or include one or more of: a target volume (e.g., three-dimensional space encompassing the tumor for delivery of radiotherapy), a dose prescription (e.g., total amount of radiation delivered to the target volume), a dose distribution (e.g., spatial arrangement of radiation in the target volume), a beam configuration (e.g., angle, energy, shape, and other arrangement of radiation beam), or a radiotherapy type (e.g., IMRT, IGRT, EBRT, SBRT, or brachytherapy), among others. With the generation, the model applier 150 may store and maintain an association between the subject 205 and the data structure 310 on the database 120 using one or more files, such as Digital Imaging and Communications in Medicine (DICOM), Health Level 7 (HL7), a portable document format (PDF), extensible markup language (XML), comma-separated values (CSV), or JavaScript Object Notation (JSON), among others.

In some embodiments, when the prompt 330 is generated for the request 320 to summarize, the model applier 150 may feed or apply the prompt 330 to the generative ML model 170 (e.g., the summarizer in the generative ML model 170). In applying, the model applier 150 may process the prompt 330, in accordance with the set of weights of the generative ML model 170. Based on applying the prompt 330 to the generative ML model 170, the model applier 150 may produce or generate at least one summary report 335. The summary report 335 may include or identify at least one of: one or more of the events corresponding to the plurality of event identifiers in the record 240; a plurality of therapy parameters defining provision of radiotherapy to an organ in the subject 250; or a plurality of characteristics defining cancer in the subject 205, among others. The summary report 335 may include content (e.g., in the form of alphanumeric characters, images, or audio) summarizing the types of information identified in the prompt 330. For instance, the summary report may include information for the user type identified in the request 320.

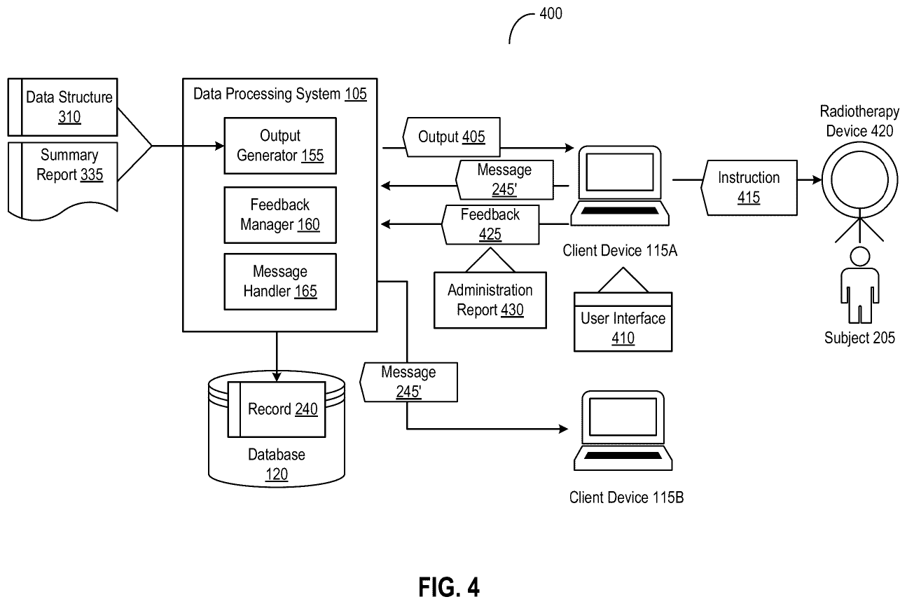

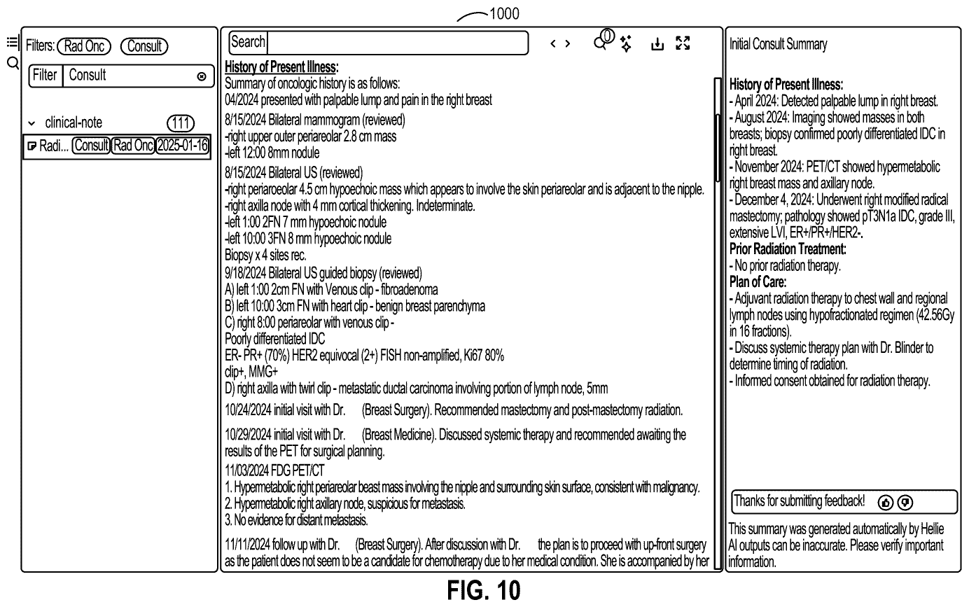

Referring now to FIG. 4, among others, depicted is a block diagram of a process 400 to produce outputs regarding radiotherapy plans in the system 100 for managing radiotherapy informatics. The process 400 may include or correspond to operations performed in the system 100 in using radiotherapy plans outputted by generative ML models. Under the process 400, the output generator 155 executing on the data processing system 105 may create, produce, or otherwise generate at least one output 405 using the data structure 310 generated for the subject 205. The output 405 may include information associated with the data structure 310 or the summary report 335. In some embodiments, the output generator 155 may create, produce, or otherwise generate the output 405 to include the data structure 310 (e.g., defining the radiotherapy timeline). In some embodiments, the output generator 155 may create, produce, or otherwise generate the output 405 to include the summary report 335. An example of the summary report 335 is depicted in FIG. 10.

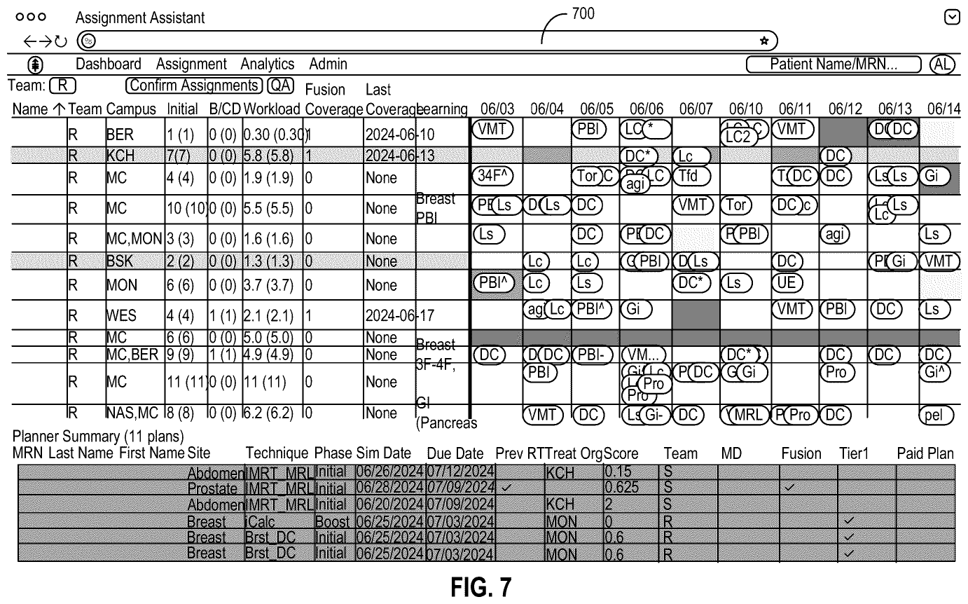

In some embodiments, the output generator 155 may create, produce, or otherwise generate the output 405 to include assignment availability information. The assignment availability information may be in response to the request 320 for clinician assignment availability for the subject 205. The request may be in connection with administration of the radiotherapy (e.g., associated with the event) by one or more clinician (e.g., an examining physician, radiologist, physicist, planner, simulation manager, pathologist, or nurse). To generate, the output generator 155 may select, retrieve, or otherwise identify a set of calendars for the clinicians. The clinician associated with the administration of the radiotherapy to the subject 205 may be identified from the record 240. For each clinician, the calendar may identify one or more time slots (e.g., within a timeframe for the administration of the radiotherapy) as one of available or unavailable for the clinician. With the identification of the set calendars, the output generator 155 may generate the assignment availability information based on the calendars for the clinicians. The assignment availability information may identify or include a set of time slots for each clinician indicated as available. An example of the analytics information is shown in FIG. 7.

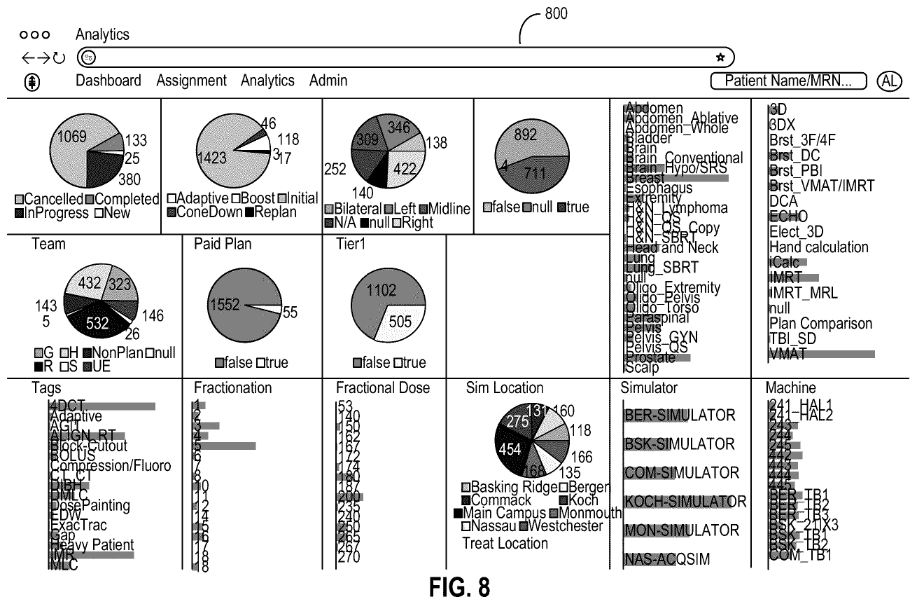

In some embodiments, the output generator 155 may create, produce, or otherwise generate the output 405 to include analytics information for the subject 205 using the record 240. The generation of the analytic information may be in response to the request 320 for analytics information for the subject 205. The analytics information may identify or include one or more of: the set of therapy parameters defining administration of radiotherapy to the organ in the subject (e.g., the radiotherapy plan 230 in at least one of the events), one or more clinical sites at which the subject is administered with radiotherapy (e.g., associated with the radiotherapy plan 230), the set of characteristics defining cancer in the subject (e.g., as identified in the pathology report 210). The analytics information may be presented using various types of visualization, such as line charts, bar charts, pie charts, histograms, scatter plots, heat maps, or geographical maps, or trees, among others. An example of the analytics information is shown in FIG. 8. In some embodiments, the output generator 155 may create, produce, or otherwise generate the information to include at least one graph using a radiotherapy plan corresponding to at least one of the set of events in the data structure 310. The graph may include or define one or more dosage values defining the administration of the radiotherapy for each portion of a target volume within the organ of the subject 205. The graph may be, for example, a dose-volume histogram (DVH) to quantify distribution of radiation doses in the target volumes and the surrounding organs at risk.

With the generation of the output 405, the output generator 155 may send, transmit, or otherwise provide the output 405 to the client device 115 (e.g., the client device 115A as depicted) for presentation via at least one user interface 410. The user interface 410 may be used to render, display, or otherwise present the information of the output 405 via the client device 115. In some embodiments, the user interface 410 may be part of a web application to be loaded by an application (e.g., a web browser) or a separate, dedicated application running on the client device 115. The user interface 410 may be used to further view or interact with the information provided in the output 405. The information displayed via the user interface 410 may include, for example, the events and relationships in the data structure 310, the set of parameters of the radiotherapy plan, the set of candidate radiotherapy plans, the timeline, or the graph, among others, or any combination thereof. In some embodiments, the output generator 155 may provide the user interface 410 for presentation via the client device 115 to select at least one from the set of candidate radiotherapy plans (e.g., for at least one of the events in the data structure 310).

Upon receipt, the client device 115 may render, display, or otherwise present the information of the output 405. When the output 405 includes the information on the data structure 310 for the subject 205, the client device 115 may present the set of parameters of the data structure 310 via the user interface elements of the user interface 410. When the output 405 includes the data structure 310, the client device 115 may present the timeline of the clinical events associated with the subject 205 on the user interface 410. In some embodiments, when the output 405 includes the analytics information, the client device 115 may display or present the visualization for the analytics information. In some embodiments, when the output 405 includes the assignment availability information, the client device 115 may display or present the assignment availability information (e.g., in calendar format). In some embodiments, when the output 405 includes the summary report 335, the client device 115 may display or present the summary report 335 via the user interface 410.

In some embodiments, when the output 405 includes the information on the set of candidate radiotherapy plans, the client device 115 may display the set of candidate radiotherapy plans along with the set of parameters for each candidate radiotherapy plan via the user interface 410. The set of candidate radiotherapy plans can be displayed upon interaction with an edge corresponding to an event for a planned administration of radiotherapy to the subject. The client device 115 may also provide user interface elements on the user interface 410 for selection of at least one of the candidate radiotherapy plans. When the output 405 includes the graph, the client device 115 may display the graph identifying dosage values for each of the target volumes within the organ affected by the cancer in the subject 205 via the user interface 410.

The client device 115 may be in communication with at least one radiotherapy device 420 to emanate, deliver, or otherwise administer the radiotherapy to the subject 205. The radiotherapy device 420 may be used to deliver the radiotherapy to the subject 205. For instance, the radiotherapy device 420 may be a linear accelerator (LINAC) device used to modulate and deliver radiation beams to treat tumors associated with the cancer in the organ of the subject 205. The radiotherapy device 420 may include one or more components, such as an electron gun to produce a stream of electrons, a waveguide to guide the electrons through the device, a bending magnet to direct the electrons toward a target, collimators to shape the beams toward the target, a control console for setting the delivery and modulation of the radiation beam, and a patient table upon which the subject 205 is situated, among others.

The client device 115 may create, produce, or otherwise generate at least one instruction 415 for the administration of the radiotherapy, in accordance with the set of parameters defined by the radiotherapy plan through the radiotherapy device 420. The instruction 415 may include the time of application of the radiotherapy, the target volume, the dose prescription, the dose distribution, the beam configuration, or the radiotherapy type, among others. In some embodiments, the client device 115 may identify one or more modifications to the data structure 310 inputted by the user of the client device 115 via the user interface 410.

The modifications may include or identify changes (e.g., by the clinician examining the subject 205) to any one or more of the parameters of the radiotherapy plan for a planned administration in the future corresponding to one of the edges in the data structure 310.

In some embodiments, the client device 115 may identify or select at least one of the radiotherapy plans based on the interaction with the one or more of the user interface elements of the user interface 410. For instance, the user of the client device 115 may select one of the candidate radiotherapy plans presented on the user interface 410 by interacting with the user interface element (e.g., a radio button) corresponding to the candidate radiotherapy plan to be selected. Upon selection, the client device 115 may extract or identify the set of parameters of the selected candidate radiotherapy plan from the radiotherapy plan to use to generate the instruction 415. The instruction 415 may include or identify data defining the set of parameters for the selected radiotherapy plan.

With the generation, the client device 115 may transmit, send, or otherwise provide the instruction 415 to the radiotherapy device 420. Upon receipt, the radiotherapy device 420 may deliver or administer the radiotherapy to the subject 205, in accordance with the set of parameters of the selected radiotherapy plan. In administering, the radiotherapy device 420 may generate a radiation beam at the specified dose prescription with the dose distribution. The radiotherapy device 420 may radiate, emit, or otherwise apply the radiation beam at the target volume (e.g., tumor in organ of the subject 205) with the beam configuration specified by the. The radiotherapy may be delivered by the radiotherapy device 420 to the subject 205 at the specified time.

In conjunction, the client device 115 may produce, create, or otherwise generate at least one feedback 425. The feedback 425 may identify or include any information generated in response to the provision of the information of the output 405 on the user interface 410 of the client device 115. For instance, the feedback 425 may include additional descriptions to events in the timeline of events made by the user of the client device 115 through the user interface elements of the user interface 410. In some embodiments, the feedback 425 may identify or include at least one administration report 430. The administration report 430 may identify or include the set of parameters of the radiotherapy administered to the subject 205. The set of parameters in the administration report 430 may differ from the set of parameters in the radiotherapy plan, due to the modifications made by the user of the client device 115. In some embodiments, the administration report 430 may identify or include the candidate radiotherapy plan selected from the set of candidate radiotherapy plans (e.g., based on the interactions with the user interface elements on the user interface 410). The administration report 430 may also include information on the delivery of the radiotherapy, such as any deviations in administration by the radiotherapy device 420, the reaction by the subject 205 to the radiotherapy, or observations by the user of the administrative device on the delivery of the radiotherapy, among others. With the generation, the client device 115 may send, transmit, or otherwise provide the feedback 425 to the data processing system 105.

The feedback manager 160 executing on the data processing system 105 may retrieve, identify, or otherwise receive the feedback 425 from the client device 115. The feedback manager 160 may parse the feedback 425 to identify the information contained therein, including the administrative report 430. With the receipt, the feedback manager 160 may update the record 240 for the subject 205 using the feedback 425. In some embodiments, the feedback manager 160 (or the data indexer 135) may create, write, or otherwise generate a set of data structures in accordance with at least one template based on the information included in the feedback 425. The template may specify or define conversion of the received feedback 425 in the original format to the data structures. The feedback manager 160 may transform or convert the received data into the data structures using the template. With the generation, the feedback manager 160 may insert, add, or otherwise include the set of data structures as part of the record 240 for the subject 205. The process of planning and delivery of radiotherapy for the subject 205 may be repeated any number of times on the data processing system 105.

In some embodiments, a sender client device 115 (e.g., the client device 115A as depicted) may create or generate at least one message 245′ to at least one recipient client device 115 (e.g., the client device 115B as depicted) via the data processing system 105. The message 245′ may be similar to the message 245 as detailed herein. The message 245′ may be associated with the subject 205. The message 245′ may contain or include content, such as a set of alphanumeric characters, image, video, or audio (e.g., inputted by the sending user) and metadata, such as sender identifier, a recipient identifier, and a timestamp identifying a time at which the message 245′ is sent, among others. The message 245′ can correspond to or include, for example, a short message service (SMS) message, a multimedia messaging service (MMS), an electronic mail, or an invocation of an API of a messaging protocol to communicate among the devices (e.g., the client devices 115A and 115B). The message 245′ may be generated by the user of the sender client device 115 in conjunction with the output 405, the instruction 415, or the feedback 425. For example, the message 245′ may be inputted by the user of the sender client device 115 in response to presentation of the output 405 or upon completion of the radiotherapy to the subject 205.

The message handler 165 may intercept, identify, or otherwise receive the message 245′ from the sender client device 115. The record 240 on the database 120 may be maintained to include messages 245 in the networked environment, including the data sources 110 or the client devices 115. With receipt of the message 245′, the message handler 165 may update the record 240 using the new message 245′ (e.g., as detailed herein in process 200). The message handler 165 may select or identify the recipient client device 115 as identified in the message 245′. With the identification, the message handler 165 may forward, send, or otherwise provide the message 245′ from the sending client device 115 to the recipient client device 115. The recipient client device 115 may retrieve, obtain, or otherwise receive the message 245′ from the sender client device 115 via the data processing system 105. Upon receipt, the recipient client device 115 may display, render, or otherwise present the message 245′ to the user.

In this manner, the data processing system 105 may maintain the records 240 of subjects 205 under evaluation for the administration of radiotherapy across a span of time to generate outputs regarding the records 240. The data processing system 105 may monitor for events across data sources 110 and multi-modal data associated with the events (e.g., in near real-time) with which to update the records 240. This monitoring and updating of records may move away from the batch, static updates, and may minimize latency between events of clinical occurrence and its reflection in the records 240. The data processing system 105 may allow for integration and harmonization of data from multiple data sources 110 with different modalities of data, thereby remedying inefficiencies due to lack of integration and siloing of the data. The data processing system 105 may also provide interoperability among the data sources 110 (e.g., including radiology, pathology, and diagnostic imaging platforms), and the client device 115 in planning and delivery radiotherapy to subjects 205. This integration and interoperability can reduce the computing resources and manual time and effort consumed in approaches that relied on users to individually access each data source to retrieve the pertinent data for radiotherapy planning. In addition, the communication of messages 245 or 245′ may provide for automated triggering of notifications of events to users of devices in the network environment.

Furthermore, the data processing system 105 can leverage generative ML model 170 that has been trained on a large set of corpuses 305, including sample radiotherapy data structures 310′ to facilitate synthesis. The data processing system 105 may employ the standardized formats of the records 240 to focus on relevant portions for generating output data structures 310 and the summary reports 335. By context-restricting prompts 330, output accuracy and usefulness of these outputs may be increased, reducing post-processing time and human review load. The data processing system 105 may use information from the record 240 derived from the data from the data sources 110 along with the initial seed parameters from the client device 115 to generate a prompt for the generative ML model 170. The radiotherapy data structure 310 outputted by the generative ML model 170 can provide useful informatics in connection with the radiotherapy to be administered to the subject 205, thereby improving the likelihood of better clinical outcomes for the cancer in the organ of the subject 205.

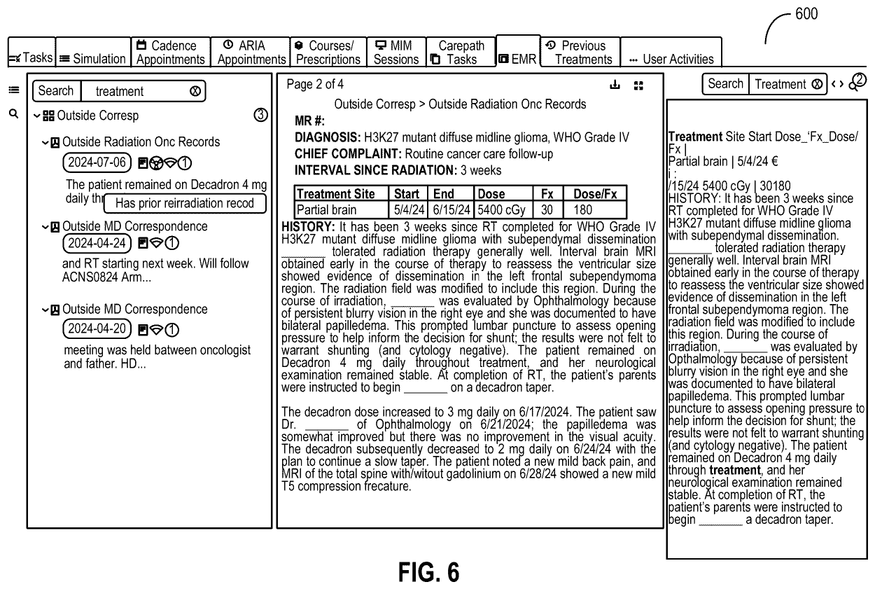

Referring now to FIG. 5, depicted is an example screenshot of a user interface 500 for presenting a timeline of clinical events associated with administration of radiotherapy to a subject. As depicted, the user interface 500 may include a user interface element containing a timeline (e.g., depicted generally along the middle) of clinical events for a given subject. The timeline may identify events, such as approval of plan, obtaining of measurements, and finalization of plans, among others. The user interface 500 may include a user interface element with additional information for each event (e.g., depicted generally along the bottom). Referring now to FIG. 6, depicted is an example screenshot of a user interface 600 for presenting medical records associated with administration of radiotherapy to a subject. As depicted, the user interface 600 may include a user interface element to navigate through a report detailing the administration of radiotherapy to a given subject (e.g., generally along the middle). The information may be presented in context with other clinical events (e.g., generally along the left).

Referring now to FIG. 7 depicts an example screenshot of a user interface 700 for presenting assignment of personnel for the administration of radiotherapy to subjects, in accordance with an illustrative embodiment. Treatment Planning Coordinators (TPCs) can assign approximately 100 newly simulated cases to approximately 80 dosimetrists per day. The assignment may entail real-time information on dosimetrists'schedule, current workload, and credential, along with the details of the new plans to be assigned. The user interface 700 may display information for assignment and may provide for assigning new cases and viewing dosimetrists load. On each row, the scheduling portion of the user interface 700 may show dosimetrists'information, such as workload, team, campus, and more.

The calendar portion shows dosimetrists'schedule (out of office, special scheduling ingested near real-time from an upstream scheduling software) along with assigned cases. The calendar captures today's date, blocks out holidays, and excludes weekends by default. The assigned cases may be denoted by an alias capturing the treatment site and technique. The cases may be color coded so that completed, in-progress, and recently assigned cases can be understood at a glance. Clicking on assigned cases may bring up a tooltip for more details, and a link to the patient details page.

Double-clicking on individual dosimetrist row may show planner summary in a tabular view to show all assigned plans and calcs that are assigned to the dosimetrist. All columns may be sortable and filterable, and the calendar date range can be adjusted. The bottom half may show the unassigned cases that are ready to be assigned. Patient information, treatment site, technique, and due date may be displayed in a table format. These cases can be dragged and dropped to a dosimetrist on the scheduler portion. This may create a draft assignment. Dosimetrists can only take on a case if they are credentialed for that treatment site and technique. The application may gray out non-credentialed dosimetrists when dragging the case, to ensure accurate assignments and prevent any patient safety issue.

Referring now to FIG. 8, depicted is an example screenshot of a user interface 800 for presenting analytics associated with the administration of radiotherapy to subjects. The data processing system 105 may integrate and pull data from upstream systems. In addition, user annotations in the workflow creates rich workflow and treatment related data to be mined. The data processing system 105 may provide visualizations of different planning parameters, as well as analysis on resource management for a user-set time range.

The planning tab may visualize multiple planning parameters and their distribution over different treatment site, technique, tags, location, machine, dose and fractionation. The pie charts and bar charts can be clicked to filter the data. On each filter, the other charts will be updated with the appropriate data counts. The data can be viewed in tabular view and the applied filters are captured in the top. Individual filters can be removed with one click, and all filters can be cleared with a reset button. The resource management tab may visualize workload distribution of dosimetrists, resource (dosimetrist) distribution over different teams (by anatomical site) and campuses. The analysis may support leadership in making hiring decisions. The data captured in the charts view can also be viewed in a table format. User-set filters may be applied across the views, and tabular view can be exported as an excel, with the date range and filter information captured in the header. The selected cohort defined by date range and filters can be easily shared with a colleague with the “Share Link” button. With proper access, the user can also view the cohort with the shared link.

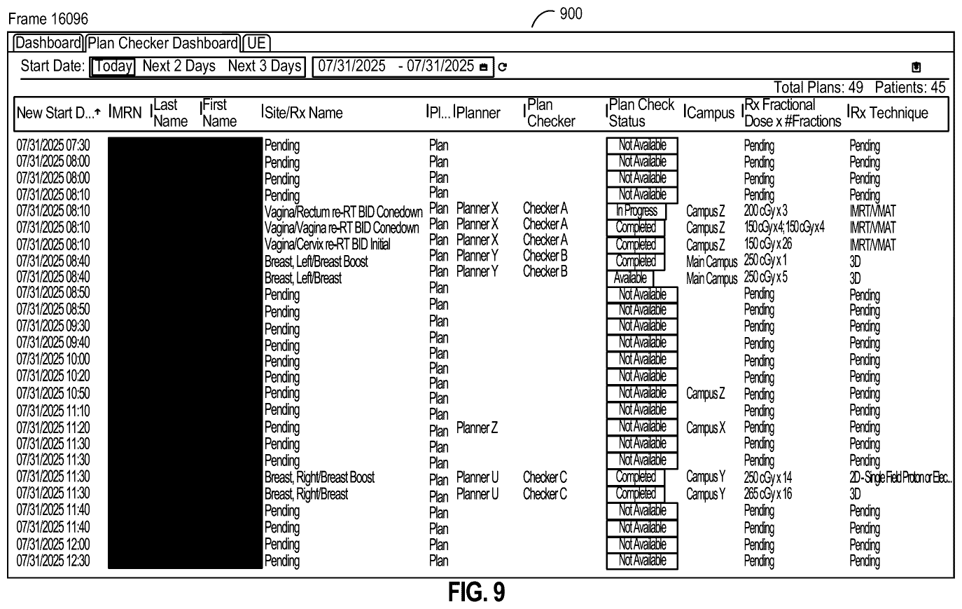

Referring now to FIG. 9, depicted is an example screenshot of a user interface 900 for presenting plan checker dashboard associated with the administration of radiotherapy to a subject. Through the user interface 900, the data processing system 105 may provide various features, such as a simulation and scheduling dashboard, smart cancellation handling (e.g., deferred logic, block expired orders), enhanced scheduling fields (e.g., creation date sort, number of days after volume/technique), indicators (e.g., fall risk, code status), and consolidated comments and notes, among others.

Referring now to FIG. 10, depicted is an example screenshot of a user interface 1000 for presenting summarized records associated with the administration of radiotherapy to a subject. The data processing system 105 may apply role-tailored prompt engineering to a summarization engine (e.g., in the generative ML model 170). Through the user interface 1000, oncologists may view comprehensive patient history, prior therapies, key imaging, or pathology data, among others. Physicists may view dose delivery details, quality assurance (QA) data, or technical machine parameters. Planners or radiation therapies may view scheduling and safety checkpoints. This may ensure each user type receives relevant insights from complex, multi-source records.

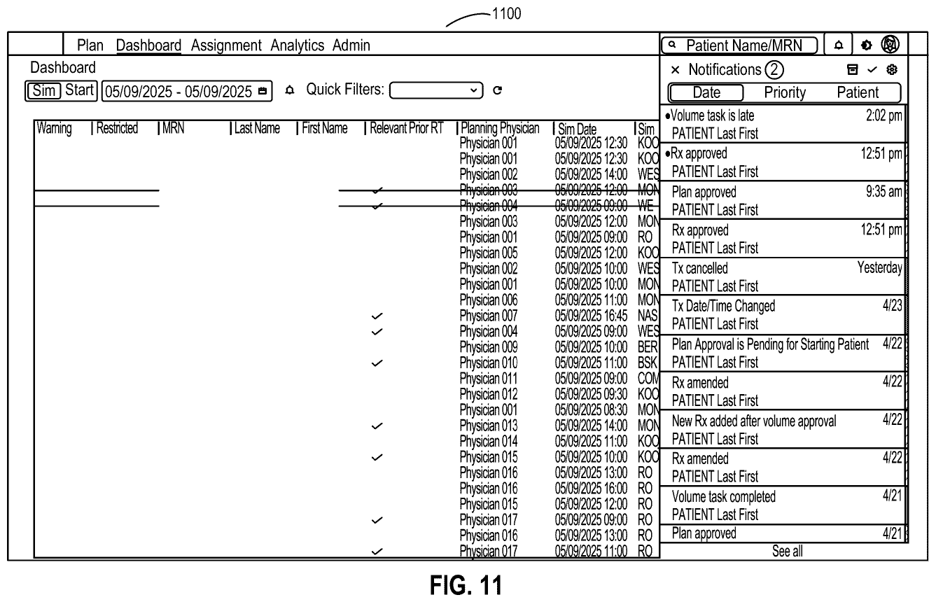

Referring now to FIG. 11, depicted is an example screenshot of a user interface 1100 for presenting notifications associated with the administration of radiotherapy to a subject. The data processing system 105 may provide notifications (e.g., a push notification). The new event-driven alerts may reduce waiting time between clinical steps. For instance, when a simulation order is cancelled, the planner can receive an instant notification through the user interface 1100, avoiding delays from email or phone updates. This may be designed to accelerate approvals, scheduling, and treatment preparation. In addition, the data processing system 105 may include integration with electronic mail. With this feature, users can email directly to the data processing system 105. The data processing system 105 may capture and organize all threads by patient and care team. The generative ML model 170 may be used to generate summarization of messages. Furthermore, the data processing system 105 may facilitate collaborative viewing and editing of the information presented through the user interface 1100. Users may be able to view a single, patient-specific communication thread instead of searching through scattered email chains. This may reduce communication friction and improve documentation consistency.

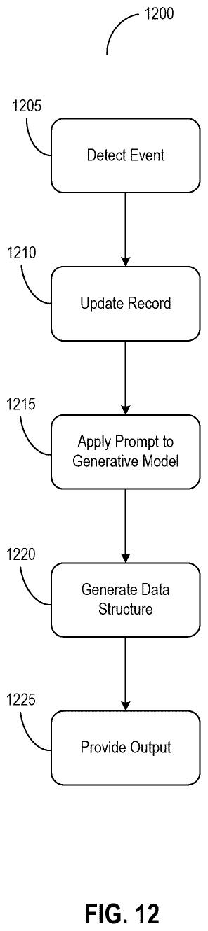

Referring now to FIG. 12, among others, depicted is a flow diagram of a method 1200 of managing radiotherapy informatics for subjects using data of disparate modalities. The method 1200 may be implemented or performed using any of the components described herein, such as the data processing system 105, or any combination thereof, or the system 1300. Under the method 1200, a computing system may detect an event in a network environment (1205). The computing system may update a record for a subject using the detected event (1210). The computing system may apply a prompt based on the record to a generative machine learning (ML) model (1215). The computing system may generate a data structure based on the application of the prompt to the generative ML model (1220). The computing system may provide an output based on the data structure (1225).

### B. Computing and Network Environment

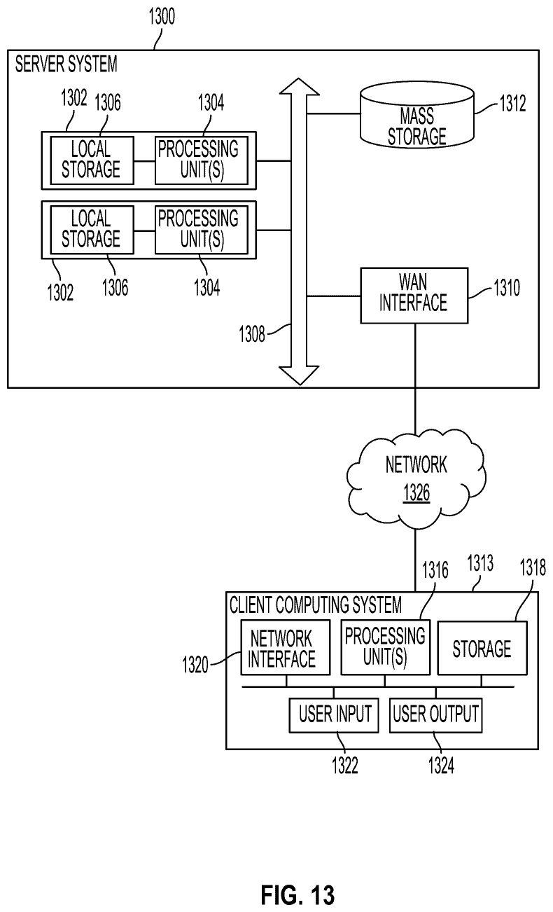

Various operations described herein can be implemented on computer systems. FIG. 13 shows a simplified block diagram of a representative server system 1300, client computing system 1314, and network 1326 usable to implement certain embodiments of the present disclosure. In various embodiments, server system 1300 or similar systems can implement services or servers described herein or portions thereof. Client computing system 1314 or similar systems can implement clients described herein. The system 1300 described herein can be similar to the server system 1300. Server system 1300 can have a modular design that incorporates a number of modules 1302 (e.g., blades in a blade server embodiment); while two modules 1302 are shown, any number can be provided. Each module 1302 can include processing unit(s) 1304 and local storage 1306.

Processing unit(s) 1304 can include a single processor, which can have one or more cores, or multiple processors. In some embodiments, processing unit(s) 1304 can include a general-purpose primary processor as well as one or more special-purpose co-processors such as graphics processors, digital signal processors, or the like. In some embodiments, some or all processing units 1304 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1304 can execute instructions stored in local storage 1306. Any type of processors in any combination can be included in processing unit(s) 1304.

Local storage 1306 can include volatile storage media (e.g., DRAM, SRAM, SDRAM, or the like) and/or non-volatile storage media (e.g., magnetic or optical disk, flash memory, or the like). Storage media incorporated in local storage 1306 can be fixed, removable or upgradeable as desired. Local storage 1306 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1304 need at runtime. The ROM can store static data and instructions that are needed by processing unit(s) 1304. The permanent storage device can be a non-volatile read-and-write memory device that can store instructions and data even when module 1302 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1306 can store one or more software programs to be executed by processing unit(s) 1304, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 1304 cause server system 1300 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1304. Software can be implemented as a single program or a collection of separate programs or program modules that interact as desired. From local storage 1306 (or non-local storage described below), processing unit(s) 1304 can retrieve program instructions to execute and data to process in order to execute various operations described above.

In some server systems 1300, multiple modules 1302 can be interconnected via a bus or other interconnect 1308, forming a local area network that supports communication between modules 1302 and other components of server system 1300. Interconnect 1308 can be implemented using various technologies including server racks, hubs, routers, and more.

A wide area network (WAN) interface 1310 can provide data communication capability between the local area network (interconnect 1308) and the network 1326, such as the Internet. Technologies can be used, including wired (e.g., Ethernet, IEEE 802.3 standards) and/or wireless technologies (e.g., Wi-Fi, IEEE 802.24 standards).

In some embodiments, local storage 1306 is intended to provide working memory for processing unit(s) 1304, providing fast access to programs and/or data to be processed while reducing traffic on interconnect 1308. Storage for larger quantities of data can be provided on the local area network by one or more mass storage subsystems 1312 that can be connected to interconnect 1308. Mass storage subsystem 1312 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1312. In some embodiments, additional data storage resources may be accessible via WAN interface 1310 (potentially with increased latency).

Server system 1300 can operate in response to requests received via WAN interface 1310. For example, one of modules 1302 can implement a supervisory function and assign discrete tasks to other modules 1302 in response to received requests. Work allocation techniques can be used. As requests are processed, results can be returned to the requester via WAN interface 1310. Such operation can generally be automated. Further, in some embodiments, WAN interface 1310 can connect multiple server systems 1300 to each other, providing scalable systems capable of managing high volumes of activity. Other techniques for managing server systems and server farms (collections of server systems that cooperate) can be used, including dynamic resource allocation and reallocation.

Server system 1300 can interact with various user-owned or user-operated devices via a wide-area network such as the Internet. An example of a user-operated device is shown in FIG. 13 as client computing system 1314. Client computing system 1314 can be implemented, for example, as a consumer device such as a smartphone, other mobile phone, tablet computer, wearable computing device (e.g., smart watch, eyeglasses), desktop computer, laptop computer, and so on.

For example, client computing system 1314 can communicate via WAN interface 1310. Client computing system 1314 can include computer components such as processing unit(s) 1316, storage device 1318, network interface 1320, user input device 1322, and user output device 1324. Client computing system 1314 can be a computing device implemented in a variety of form factors, such as a desktop computer, laptop computer, tablet computer, smartphone, other mobile computing device, wearable computing device, or the like.

Processing unit(s) 1316 and storage device 1318 can be similar to processing unit(s) 1304 and local storage 1306 described above. Suitable devices can be selected based on the demands to be placed on client computing system 1314; for example, client computing system 1314 can be implemented as a “thin” client with limited processing capability or as a high-powered computing device. Client computing system 1314 can be provisioned with program code executable by processing unit(s) 1316 to enable various interactions with server system 1300.

Network interface 1320 can provide a connection to the network 1326, such as a wide area network (e.g., the Internet) to which WAN interface 1310 of server system 1300 is also connected. In various embodiments, network interface 1320 can include a wired interface (e.g., Ethernet) and/or a wireless interface implementing various RF data communication standards such as Wi-Fi, Bluetooth, or cellular data network standards (e.g., 3G, 4G, 5G, 6G, LTE, etc.).

User input device 1322 can include any device (or devices) via which a user can provide signals to client computing system 1314; client computing system 1314 can interpret the signals as indicative of particular user requests or information. In various embodiments, user input device 1322 can include any or all of a keyboard, touch pad, touch screen, mouse or other pointing device, scroll wheel, click wheel, dial, button, switch, keypad, microphone, and so on.

User output device 1324 can include any device via which client computing system 1314 can provide information to a user. For example, user output device 1324 can include a display to present images generated by or delivered to client computing system 1314. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1324 can be provided in addition to or instead of a display. Examples include indicator lights, speakers, tactile “display” devices, printers, and so on.

Some embodiments include electronic components, such as microprocessors, storage and memory that store computer program instructions in a computer-readable storage medium. Many of the features described in this specification can be implemented as processes that are specified as a set of program instructions encoded on a computer-readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1304 and 1316 can provide various functionality for server system 1300 and client computing system 1314, including any of the functionality described herein as being performed by a server or client, or other functionality.

It will be appreciated that server system 1300 and client computing system 1314 are illustrative and that variations and modifications are possible. Computer systems used in connection with embodiments of the present disclosure can have other capabilities not specifically described here. Further, while server system 1300 and client computing system 1314 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to the specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may reference specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer-readable storage media; suitable media include magnetic disk or tape, optical storage media such as compact disk (CD) or DVD (digital versatile disk), flash memory, and other non-transitory media. Computer-readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

What is claimed is:

1. A method of generating data structures for data structures for events detected across data sources in network environments, comprising:
detecting, by one or more processors, from one or more data sources in a network environment, an event associated with provision of radiation to a subject;
updating, by the one or more processors, using an event identifier and a timestamp corresponding to the event, a record comprising (i) a plurality of event identifiers for a corresponding plurality of events associated with the subject and (ii) a plurality of timestamps corresponding to the plurality of events on a database;
generating, by the one or more processors, a prompt based on at least a portion of the record on the database;
providing, by the one or more processors, the prompt to a generative machine learning (ML) model, wherein the generative ML model is established using a plurality of corpuses, each of the plurality of corpuses including:
(i) a respective record of a corresponding subject identifying (a) a respective plurality of event identifiers for a respective plurality of events associated with provision of radiation in the corresponding subject and (b) a respective plurality of timestamps corresponding to the respective plurality of event identifiers, and
(ii) a respective data structure identifying (a) a plurality of nodes corresponding to the respective plurality of event identifiers and (b) a respective plurality of edges each defining a relationship between a corresponding pair of the plurality of nodes;
generating, by the one or more processors, based on applying the prompt to the generative ML model, a data structure comprising (i) a plurality of nodes corresponding to a plurality of event identifiers and (ii) a plurality of edges each defining a relationship between a corresponding pair of the plurality of nodes; and
providing, by the one or more processors, via a user interface, an output based on the data structure for the subject.

2. The method of claim 1, wherein detecting the event further comprises receiving, via the user interface, a report comprising at least one of:
(i) a plurality of radiation parameters defining the provision of the radiation to an organ of the subject, wherein the plurality of radiation parameters associated with the radiation comprises at least one of a target volume, a dose of radiation, a dose distribution, a beam configuration, or a radiation type, wherein the radiation comprises at least one of intensity-modulated radiation therapy (IMRT), external beam radiation therapy (EBRT), stereotactic body radiation therapy (SBRT), image-guided radiation therapy (IGRT), or brachytherapy; or
(ii) a plurality of characteristics defining a condition in the subject, wherein the plurality of characteristics defining cancer in the subject comprises at least one of a cancer type, a tumor classification, a tumor size, a tumor appearance, or a tumor grade and
wherein updating the record further comprising updating the record for the subject on the database using the report.

3. The method of claim 1, further comprising:
retrieving, by the one or more processors, from one or more data sources, data associated with the plurality of event identifiers for the corresponding plurality of events and a corresponding plurality of timestamps,
wherein the plurality of events comprises at least one of approval of radiation, generation of radiation simulation, provision of radiation, subject diagnosis, or an acquisition of a biomedical image,
wherein the biomedical image is in accordance with one of a plurality of imaging modalities including a whole slide imaging (WSI) modality, a computed tomography (CT) modality, a magnetic resonance imaging (MRI) modality, a positron emission tomography (PET) modality, or an x-ray imaging modality;
generating, by the one or more processors, for storage on the database, the record, in accordance with a template based on the data retrieved from the one or more data sources.

4. The method of claim 1, further comprising:
providing, by the one or more processors, for presentation, the user interface comprising one or more user interface elements to select at least one of a plurality of radiation plans for the subjects, each of the plurality of radiation plans identifying a corresponding plurality of radiation parameters defining provision of a respective radiation to an organ in the subject at a corresponding time; and
selecting, by the one or more processors, for presentation via the user interface, a radiation plan from the plurality of radiation plans based on interaction with the one or more user interface elements.

5. The method of claim 1, wherein generating the data structure comprising generating the data structure defining a timeline to include the plurality of nodes corresponding to the respective plurality of event identifiers, each of the plurality of nodes configured to provide information on a corresponding event of the plurality of events responsive to interaction.

6. The method of claim 1, further comprising:
generating, by the one or more processors, a second prompt in accordance with a template for a user type of the user interface and using at least a portion of the record for the subject;
providing, by the one or more processors, the second prompt to the generative ML model; to generate a report identifying at least one of (i) one or more of the plurality of events corresponding to the plurality of event identifiers (ii) a plurality of therapy parameters defining provision of radiation to an organ in the subject or(iii) a plurality of characteristics defining cancer in the subject; and
providing, by the one or more processors, via the user interface, a second output based on the report.

7. The method of claim 1, further comprising:
identifying, by the one or more processors, for provision of the radiation to the subject, a plurality of calendars for a plurality of clinicians, each calendar of the plurality of calendars identifying a plurality of time slots indicating as one of available or unavailable for a corresponding clinician of the plurality of clinicians; and
generating, by the one or more processors, for presentation via the user interface, assignment availability information based on the plurality of calendars for a plurality of clinicians.

8. The method of claim 1, wherein providing the output further comprises generating, using a radiation plan corresponding to at least one of the plurality of events, a graph to identify one or more dosage values defining the provision of the radiation for each volume of a plurality of volumes in an organ of the subject.

9. The method of claim 1, further comprising:
maintaining, by the one or more processors, the record to include a plurality of messages associated with the provision of the radiation to the subject from one or more data sources; and
receiving, by the one or more processors, from a first client device in the network environment, a message associated with the provision of the radiation to the subject; and
providing, by the one or more processors, to a second client device for presentation, a notification identifying the message.

10. The method of claim 1, wherein the record further comprises at least one of (i) a report including a first plurality of radiation parameters defining radiation administered to an organ in the subject, or (ii) a simulated radiation plan including a second plurality of radiation parameters defining provision of radiation to the organ in the subject,
wherein the subject is at risk of or diagnosed with cancer comprising at least one of lung cancer, brain cancer, head and neck cancer, colon cancer, rectal cancer, uterine cancer, endometrial cancer, stomach cancer, ovarian cancer, cervical cancer, bladder cancer, or breast cancer.

11. A system for generating data structures for data structures for events detected across data sources in network environments, comprising:
one or more processors coupled with memory, configured to:
detect, from one or more data sources in a network environment, an event associated with provision of radiation to a subject;
update, using an event identifier and a timestamp corresponding to the event, a record comprising (i) a plurality of event identifiers for a corresponding plurality of events associated with the subject and (ii) a plurality of timestamps corresponding to the plurality of events on a database;
generate a prompt based on at least a portion of the record on the database;
provide the prompt to a generative machine learning (ML) model, wherein the generative ML model is established using a plurality of corpuses, each of the plurality of corpuses including:
(i) a respective record of a corresponding subject identifying (a) a respective plurality of event identifiers for a respective plurality of events associated with provision of radiation in the corresponding subject and (b) a respective plurality of timestamps corresponding to the respective plurality of event identifiers, and
(ii) a respective data structure identifying (a) a plurality of nodes corresponding to the respective plurality of event identifiers and (b) a respective plurality of edges each defining a relationship between a corresponding pair of the plurality of nodes;
generate, based on applying the prompt to the generative ML model, a data structure comprising (i) a plurality of nodes corresponding to a plurality of event identifiers and (ii) a plurality of edges each defining a relationship between a corresponding pair of the plurality of nodes; and
provide, via a user interface, an output based on the data structure for the subject.

12. The system of claim 11, wherein the one or more processors are further configured to:
receive, via the user interface, a report comprising at least one of:
(i) a plurality of radiation parameters defining the provision of the radiation to an organ of the subject, wherein the plurality of radiation parameters associated with the radiation comprises at least one of a target volume, a dose of radiation, a dose distribution, a beam configuration, or a radiation type, wherein the radiation comprises at least one of intensity-modulated radiation therapy (IMRT), external beam radiation therapy (EBRT), stereotactic body radiation therapy (SBRT), image-guided radiation therapy (IGRT), or brachytherapy; or
(ii) a plurality of characteristics defining a condition in the subject, wherein the plurality of characteristics defining cancer in the subject comprises at least one of a cancer type, a tumor classification, a tumor size, a tumor appearance, or a tumor grade and
update the record for the subject on the database using the report.

13. The system of claim 11, wherein the one or more processors are further configured to:
retrieve, from one or more data sources, data associated with the plurality of event identifiers for the corresponding plurality of events and a corresponding plurality of timestamps,
wherein the plurality of events comprises at least one of approval of radiation, generation of radiation simulation, provision of radiation, subject diagnosis, or an acquisition of a biomedical image,
wherein the biomedical image is in accordance with one of a plurality of imaging modalities including a whole slide imaging (WSI) modality, a computed tomography (CT) modality, a magnetic resonance imaging (MRI) modality, a positron emission tomography (PET) modality, or an x-ray imaging modality;
generate, for storage on the database, the record, in accordance with a template based on the data retrieved from the one or more data sources.

14. The system of claim 11, wherein the one or more processors are further configured to:
provide, for presentation, the user interface comprising one or more user interface elements to select at least one of a plurality of radiation plans for the subjects, each of the plurality of radiation plans identifying a corresponding plurality of radiation parameters defining provision of a respective radiation to an organ in the subject at a corresponding time; and
select, for presentation via the user interface, a radiation plan from the plurality of radiation plans based on interaction with the one or more user interface elements.

15. The system of claim 11, wherein the one or more processors are further configured to generate the data structure defining a timeline to include the plurality of nodes corresponding to the respective plurality of event identifiers, each of the plurality of nodes configured to provide information on a corresponding event of the plurality of events responsive to interaction.

16. The system of claim 11, wherein the one or more processors are further configured to
generate a second prompt in accordance with a template for a user type of the user interface and using at least a portion of the record for the subject;
provide the second prompt to the generative ML model; to generate a report identifying at least one of (i) one or more of the plurality of events corresponding to the plurality of event identifiers (ii) a plurality of therapy parameters defining provision of radiation to an organ in the subject or(iii) a plurality of characteristics defining cancer in the subject; and
provide, via the user interface, a second output based on the report.

17. The system of claim 11, wherein the one or more processors are further configured to
identify, for provision of the radiation to the subject, a plurality of calendars for a plurality of clinicians, each calendar of the plurality of calendars identifying a plurality of time slots indicating as one of available or unavailable for a corresponding clinician of the plurality of clinicians; and
generate, for presentation via the user interface, assignment availability information based on the plurality of calendars for a plurality of clinicians.

18. The system of claim 11, wherein the one or more processors are further configured to generate, using a radiation plan corresponding to at least one of the plurality of events, a graph to identify one or more dosage values defining the provision of the radiation for each volume of a plurality of volumes in an organ of the subject.

19. The system of claim 11, wherein the one or more processors are further configured to:
maintain the record to include a plurality of messages associated with the provision of the radiation to the subject from one or more data sources; and
receive, from a first client device in the network environment, a message associated with the provision of the radiation to the subject; and
provide, to a second client device for presentation, a notification identifying the message.

20. The system of claim 11, wherein the record further comprises at least one of (i) a report including a first plurality of radiation parameters defining radiation administered to an organ in the subject, or (ii) a simulated radiation plan including a second plurality of radiation parameters defining provision of radiation to the organ in the subject,
wherein the subject is at risk of or diagnosed with cancer comprising at least one of lung cancer, brain cancer, head and neck cancer, colon cancer, rectal cancer, uterine cancer, endometrial cancer, stomach cancer, ovarian cancer, cervical cancer, bladder cancer, or breast cancer.
